(19) United States
(12) Patent Application Publication
(10) Pub. No.: US 2025/0339441 A1
(43) Pub. Date: Nov. 6, 2025

(54) METHODS OF TREATING CANCER

(71) Applicant: FOGHORN THERAPEUTICS INC., Cambridge, MA (US)

(72) Inventors: Virna SCHUCK, Parsippany, NJ (US); Samuel AGRESTA, Lexington, MA (US); Sarah REILLY, Boston, MA (US); Martin F. HENTEMANN, Carlisle, MA (US)

(21) Appl. No.: 18/855,015

(22) PCT Filed: Apr. 7, 2023
(86) PCT No.: PCT/US2023/017829

§ 371 Date: Oct. 8, 2024

Related U.S. Application Data
(60) Provisional application No. 63/329,048, filed on Apr. 8, 2022.

(51) Int. Cl. A61K 31/5377; A61P 35/00

(52) U.S. Cl. CPC A61K 31/5377; A61P 35/00

(57) ABSTRACT

The present disclosure features methods, e.g., dosing regimens, of treating cancer with SMARCA4/SMARCA2 ATPase inhibitors.

### SEQUENCE LISTING

The instant application contains a Sequence Listing which has been submitted electronically in XML format and is hereby incorporated by reference in its entirety. Said XML copy, created on May 31, 2023, is named 51121-077WO2_Sequence_Listing_5_31_23.xml and is 19,207 bytes in size.

### BACKGROUND

The present disclosure relates to compounds and methods useful for modulating BRG1- or BRM-associated factors (BAF) complexes. In particular, the present disclosure relates to compounds and methods useful for treatment of disorders associated with BAF complex function, such as cancer.

Chromatin regulation is essential for gene expression, and ATP-dependent chromatin remodeling is a mechanism by which such gene expression occurs. The human Switch/Sucrose Non-Fermentable (SWI/SNF) chromatin remodeling complex, also known as BAF complex, has two SWI2-like ATPases known as BRG1 (Brahma-related gene-1) and BRM (Brahma). The transcription activator BRG1, also known as ATP-dependent chromatin remodeler SMARCA4, is encoded by the SMARCA4 gene on chromosome 19. BRG1 is overexpressed in some cancer tumors and is needed for cancer cell proliferation. BRM, also known as probable global transcription activator SNF2L2 and/or ATP-dependent chromatin remodeler SMARCA2, is encoded by the SMARCA2 gene on chromosome 9 and has been shown to be essential for tumor cell growth in cells characterized by loss of BRG1 function mutations. Deactivation of BRG and/or BRM results in downstream effects in cells, including cell cycle arrest and tumor suppression.

### SUMMARY

The present disclosure features methods of administering a compound, N-(1-((4-(6-(2,6-dimethylmorpholino)pyridin-2-yl)thiazol-2-yl)amino)-3-methoxy-1-oxopropan-2-yl)-1-(methylsulfonyl)-1H-pyrrole-3-carboxamide, or a pharmaceutically acceptable salt thereof, to a subject in need thereof.

In an aspect, the invention provides a method of treating cancer in a subject in need thereof, the method including administering to the subject an effective amount of a compound, N-(1-((4-(6-(2,6-dimethylmorpholino)pyridin-2-yl)thiazol-2-yl)amino)-3-methoxy-1-oxopropan-2-yl)-1-(methylsulfonyl)-1H-pyrrole-3-carboxamide, or a pharmaceutically acceptable salt thereof, at least once daily for six to eight days, followed by six to eight days without administration of the compound, or a pharmaceutically acceptable salt thereof.

In an aspect, the invention provides a method of treating cancer in a subject in need thereof, the method including administering to the subject an effective amount of a compound, N-(1-((4-(6-(2,6-dimethylmorpholino)pyridin-2-yl)thiazol-2-yl)amino)-3-methoxy-1-oxopropan-2-yl)-1-(methylsulfonyl)-1H-pyrrole-3-carboxamide, or a pharmaceutically acceptable salt thereof, at least once daily for thirteen to fifteen days, followed by six to eight days without administration of the compound, or a pharmaceutically acceptable salt thereof.

In an aspect, the invention provides a method of treating cancer in a subject in need thereof, the method including administering to the subject an effective amount of a compound, N-(1-((4-(6-(2,6-dimethylmorpholino)pyridin-2-yl)thiazol-2-yl)amino)-3-methoxy-1-oxopropan-2-yl)-1-(methylsulfonyl)-1H-pyrrole-3-carboxamide, or a pharmaceutically acceptable salt thereof, at least once daily for six to fifteen days, followed by six to eight days without administration of the compound, or a pharmaceutically acceptable salt thereof.

In an aspect, the invention provides a method of treating cancer in a subject in need thereof, the method including administering to the subject an effective amount of a compound, N-(1-((4-(6-(2,6-dimethylmorpholino)pyridin-2-yl)thiazol-2-yl)amino)-3-methoxy-1-oxopropan-2-yl)-1-(methylsulfonyl)-1H-pyrrole-3-carboxamide, or a pharmaceutically acceptable salt thereof, at least once daily for seven days, followed by seven days without administration of the compound, or a pharmaceutically acceptable salt thereof.

In an aspect, the invention provides a method of treating cancer in a subject in need thereof, the method including administering to the subject an effective amount of a compound, N-(1-((4-(6-(2,6-dimethylmorpholino)pyridin-2-yl)thiazol-2-yl)amino)-3-methoxy-1-oxopropan-2-yl)-1-(methylsulfonyl)-1H-pyrrole-3-carboxamide, or a pharmaceutically acceptable salt thereof, at least once daily for fourteen days, followed by seven days without administration of the compound, or a pharmaceutically acceptable salt thereof.

In an aspect, the invention provides a method of treating cancer in a subject in need thereof, the method including administering to the subject an effective amount of a compound, N-(1-((4-(6-(2,6-dimethylmorpholino)pyridin-2-yl)thiazol-2-yl)amino)-3-methoxy-1-oxopropan-2-yl)-1-(methylsulfonyl)-1H-pyrrole-3-carboxamide, or a pharmaceutically acceptable salt thereof, at least once daily for seven to fourteen days, followed by seven days without administration of the compound, or a pharmaceutically acceptable salt thereof.

In an aspect, the invention provides a method of decreasing the level and/or activity of BRG1 and/or BRM in a subject, the method including administering to the subject an effective amount of a compound, N-(1-((4-(6-(2,6-dimethylmorpholino)pyridin-2-yl)thiazol-2-yl)amino)-3-methoxy-1-oxopropan-2-yl)-1-(methylsulfonyl)-1H-pyrrole-3-carboxamide, or a pharmaceutically acceptable salt thereof, at least once daily for six to eight days, followed by six to eight days without administration of the compound, or a pharmaceutically acceptable salt thereof.

In an aspect, the invention provides a method of decreasing the level and/or activity of BRG1 and/or BRM in a subject, the method including administering to the subject an effective amount of a compound, N-(1-((4-(6-(2,6-dimethylmorpholino)pyridin-2-yl)thiazol-2-yl)amino)-3-methoxy-1-oxopropan-2-yl)-1-(methylsulfonyl)-1H-pyrrole-3-carboxamide, or a pharmaceutically acceptable salt thereof, at least once daily for thirteen to fifteen days, followed by six to eight days without administration of the compound, or a pharmaceutically acceptable salt thereof.

In an aspect, the invention provides a method of decreasing the level and/or activity of BRG1 and/or BRM in a subject, the method including administering to the subject an effective amount of a compound, N-(1-((4-(6-(2,6-dimethylmorpholino)pyridin-2-yl)thiazol-2-yl)amino)-3-methoxy-1-oxopropan-2-yl)-1-(methylsulfonyl)-1H-pyrrole-3-carboxamide, or a pharmaceutically acceptable salt thereof, at least once daily for six to fifteen days, followed by six to eight days without administration of the compound, or a pharmaceutically acceptable salt thereof.

In an aspect, the invention provides a method of decreasing the level and/or activity of BRG1 and/or BRM in a subject, the method including administering to the subject an effective amount of a compound, N-(1-((4-(6-(2,6-dimethylmorpholino)pyridin-2-yl)thiazol-2-yl)amino)-3-methoxy-1-oxopropan-2-yl)-1-(methylsulfonyl)-1H-pyrrole-3-carboxamide, or a pharmaceutically acceptable salt thereof, at least once daily for seven days, followed by seven days without administration of the compound, or a pharmaceutically acceptable salt thereof.

In an aspect, the invention provides a method of decreasing the level and/or activity of BRG1 and/or BRM in a subject, the method including administering to the subject an effective amount of a compound, N-(1-((4-(6-(2,6-dimethylmorpholino)pyridin-2-yl)thiazol-2-yl)amino)-3-methoxy-1-oxopropan-2-yl)-1-(methylsulfonyl)-1H-pyrrole-3-carboxamide, or a pharmaceutically acceptable salt thereof, at least once daily for fourteen days, followed by seven days without administration of the compound, or a pharmaceutically acceptable salt thereof.

In an aspect, the invention provides a method of decreasing the level and/or activity of BRG1 and/or BRM in a subject, the method including administering to the subject an effective amount of a compound, N-(1-((4-(6-(2,6-dimethylmorpholino)pyridin-2-yl)thiazol-2-yl)amino)-3-methoxy-1-oxopropan-2-yl)-1-(methylsulfonyl)-1H-pyrrole-3-carboxamide, or a pharmaceutically acceptable salt thereof, at least once daily for seven to fourteen days, followed by seven days without administration of the compound, or a pharmaceutically acceptable salt thereof.

In some embodiments, the subject has cancer.

In an aspect, the invention provides a method of treating a BAF complex-related disorder in a subject in need thereof, the method including administering to the subject an effective amount of a compound, N-(1-((4-(6-(2,6-dimethylmorpholino)pyridin-2-yl)thiazol-2-yl)amino)-3-methoxy-1-oxopropan-2-yl)-1-(methylsulfonyl)-1H-pyrrole-3-carboxamide, or a pharmaceutically acceptable salt thereof, at least once daily for six to eight days, followed by six to eight days without administration of the compound, or a pharmaceutically acceptable salt thereof.

In an aspect, the invention provides a method of treating a BAF complex-related disorder in a subject in need thereof, the method including administering to the subject an effective amount of a compound, N-(1-((4-(6-(2,6-dimethylmorpholino)pyridin-2-yl)thiazol-2-yl)amino)-3-methoxy-1-oxopropan-2-yl)-1-(methylsulfonyl)-1H-pyrrole-3-carboxamide, or a pharmaceutically acceptable salt thereof, at least once daily for thirteen to fifteen days, followed by six to eight days without administration of the compound, or a pharmaceutically acceptable salt thereof.

In an aspect, the invention provides a method of treating a BAF complex-related disorder in a subject in need thereof, the method including administering to the subject an effective amount of a compound, N-(1-((4-(6-(2,6-dimethylmorpholino)pyridin-2-yl)thiazol-2-yl)amino)-3-methoxy-1-oxopropan-2-yl)-1-(methylsulfonyl)-1H-pyrrole-3-carboxamide, or a pharmaceutically acceptable salt thereof, at least once daily for six to fifteen days, followed by six to eight days without administration of the compound, or a pharmaceutically acceptable salt thereof.

In an aspect, the invention provides a method of treating a BAF complex-related disorder in a subject in need thereof, the method including administering to the subject an effective amount of a compound, N-(1-((4-(6-(2,6-dimethylmorpholino)pyridin-2-yl)thiazol-2-yl)amino)-3-methoxy-1-oxopropan-2-yl)-1-(methylsulfonyl)-1H-pyrrole-3-carboxamide, or a pharmaceutically acceptable salt thereof, at least once daily for seven days, followed by seven days without administration of the compound, or a pharmaceutically acceptable salt thereof.

In an aspect, the invention provides a method of treating a BAF complex-related disorder in a subject in need thereof, the method including administering to the subject an effective amount of a compound, N-(1-((4-(6-(2,6-dimethylmorpholino)pyridin-2-yl)thiazol-2-yl)amino)-3-methoxy-1-oxopropan-2-yl)-1-(methylsulfonyl)-1H-pyrrole-3-carboxamide, or a pharmaceutically acceptable salt thereof, at least once daily for fourteen days, followed by seven days without administration of the compound, or a pharmaceutically acceptable salt thereof.

In an aspect, the invention provides a method of treating a BAF complex-related disorder in a subject in need thereof, the method including administering to the subject an effective amount of a compound, N-(1-((4-(6-(2,6-dimethylmorpholino)pyridin-2-yl)thiazol-2-yl)amino)-3-methoxy-1-oxopropan-2-yl)-1-(methylsulfonyl)-1H-pyrrole-3-carboxamide, or a pharmaceutically acceptable salt thereof, at least once daily for seven to fourteen days, followed by seven days without administration of the compound, or a pharmaceutically acceptable salt thereof.

In an aspect, the invention provides a method of treating a disorder related to a BRG1 loss of function mutation in a subject in need thereof, the method including administering to the subject an effective amount of a compound, N-(1-((4-(6-(2,6-dimethylmorpholino)pyridin-2-yl)thiazol-2-yl)amino)-3-methoxy-1-oxopropan-2-yl)-1-(methylsulfonyl)-1H-pyrrole-3-carboxamide, or a pharmaceutically acceptable salt thereof, at least once daily for six to eight days, followed by six to eight days without administration of the compound, or a pharmaceutically acceptable salt thereof.

In an aspect, the invention provides a method of treating a disorder related to a BRG1 loss of function mutation in a subject in need thereof, the method including administering to the subject an effective amount of a compound, N-(1-((4-(6-(2,6-dimethylmorpholino)pyridin-2-yl)thiazol-2-yl)amino)-3-methoxy-1-oxopropan-2-yl)-1-(methylsulfonyl)-1H-pyrrole-3-carboxamide, or a pharmaceutically acceptable salt thereof, at least once daily for thirteen to fifteen days, followed by six to eight days without administration of the compound, or a pharmaceutically acceptable salt thereof.

In an aspect, the invention provides a method of treating a disorder related to a BRG1 loss of function mutation in a subject in need thereof, the method including administering to the subject an effective amount of a compound, N-(1-((4-(6-(2,6-dimethylmorpholino)pyridin-2-yl)thiazol-2-yl)amino)-3-methoxy-1-oxopropan-2-yl)-1-(methylsulfonyl)-1H-pyrrole-3-carboxamide, or a pharmaceutically acceptable salt thereof, at least once daily for six to fifteen days, followed by six to eight days without administration of the compound, or a pharmaceutically acceptable salt thereof.

In an aspect, the invention provides a method of treating a disorder related to a BRG1 loss of function mutation in a subject in need thereof, the method including administering to the subject an effective amount of a compound, N-(1-((4-(6-(2,6-dimethylmorpholino)pyridin-2-yl)thiazol-2-yl)amino)-3-methoxy-1-oxopropan-2-yl)-1-(methylsulfonyl)-1H-pyrrole-3-carboxamide, or a pharmaceutically acceptable salt thereof, at least once daily for seven days, followed by seven days without administration of the compound, or a pharmaceutically acceptable salt thereof.

In an aspect, the invention provides a method of treating a disorder related to a BRG1 loss of function mutation in a subject in need thereof, the method including administering to the subject an effective amount of a compound, N-(1-((4-(6-(2,6-dimethylmorpholino)pyridin-2-yl)thiazol-2-yl)amino)-3-methoxy-1-oxopropan-2-yl)-1-(methylsulfonyl)-1H-pyrrole-3-carboxamide, or a pharmaceutically acceptable salt thereof, at least once daily for fourteen days, followed by seven days without administration of the compound, or a pharmaceutically acceptable salt thereof.

In an aspect, the invention provides a method of treating a disorder related to a BRG1 loss of function mutation in a subject in need thereof, the method including administering to the subject an effective amount of a compound, N-(1-((4-(6-(2,6-dimethylmorpholino)pyridin-2-yl)thiazol-2-yl)amino)-3-methoxy-1-oxopropan-2-yl)-1-(methylsulfonyl)-1H-pyrrole-3-carboxamide, or a pharmaceutically acceptable salt thereof, at least once daily for seven to fourteen days, followed by seven days without administration of the compound, or a pharmaceutically acceptable salt thereof.

In some embodiments, the disorder is cancer.

In an aspect, the invention provides a method of decreasing the level and/or activity of a BAF complex in a cell of a subject, the method including administering to the subject an effective amount of a compound, N-(1-((4-(6-(2,6-dimethylmorpholino)pyridin-2-yl)thiazol-2-yl)amino)-3-methoxy-1-oxopropan-2-yl)-1-(methylsulfonyl)-1H-pyrrole-3-carboxamide, or a pharmaceutically acceptable salt thereof, at least once daily for six to eight days, followed by six to eight days without administration of the compound, or a pharmaceutically acceptable salt thereof.

In an aspect, the invention provides a method of decreasing the level and/or activity of a BAF complex in a cell of a subject, the method including administering to the subject an effective amount of a compound, N-(1-((4-(6-(2,6-dimethylmorpholino)pyridin-2-yl)thiazol-2-yl)amino)-3-methoxy-1-oxopropan-2-yl)-1-(methylsulfonyl)-1H-pyrrole-3-carboxamide, or a pharmaceutically acceptable salt thereof, at least once daily for thirteen to fifteen days, followed by six to eight days without administration of the compound, or a pharmaceutically acceptable salt thereof.

In an aspect, the invention provides a method of decreasing the level and/or activity of a BAF complex in a cell of a subject, the method including administering to the subject an effective amount of a compound, N-(1-((4-(6-(2,6-dimethylmorpholino)pyridin-2-yl)thiazol-2-yl)amino)-3-methoxy-1-oxopropan-2-yl)-1-(methylsulfonyl)-1H-pyrrole-3-carboxamide, or a pharmaceutically acceptable salt thereof, at least once daily for six to fifteen days, followed by six to eight days without administration of the compound, or a pharmaceutically acceptable salt thereof.

In an aspect, the invention provides a method of decreasing the level and/or activity of a BAF complex in a cell of a subject, the method including administering to the subject an effective amount of a compound, N-(1-((4-(6-(2,6-dimethylmorpholino)pyridin-2-yl)thiazol-2-yl)amino)-3-methoxy-1-oxopropan-2-yl)-1-(methylsulfonyl)-1H-pyrrole-3-carboxamide, or a pharmaceutically acceptable salt thereof, at least once daily for seven days, followed by seven days without administration of the compound, or a pharmaceutically acceptable salt thereof.

In an aspect, the invention provides a method of decreasing the level and/or activity of a BAF complex in a cell of a subject, the method including administering to the subject an effective amount of a compound, N-(1-((4-(6-(2,6-dimethylmorpholino)pyridin-2-yl)thiazol-2-yl)amino)-3-methoxy-1-oxopropan-2-yl)-1-(methylsulfonyl)-1H-pyrrole-3-carboxamide, or a pharmaceutically acceptable salt thereof, at least once daily for fourteen days, followed by seven days without administration of the compound, or a pharmaceutically acceptable salt thereof.

In an aspect, the invention provides a method of decreasing the level and/or activity of a BAF complex in a cell of a subject, the method including administering to the subject an effective amount of a compound, N-(1-((4-(6-(2,6-dimethylmorpholino)pyridin-2-yl)thiazol-2-yl)amino)-3-methoxy-1-oxopropan-2-yl)-1-(methylsulfonyl)-1H-pyrrole-3-carboxamide, or a pharmaceutically acceptable salt thereof, at least once daily for seven to fourteen days, followed by seven days without administration of the compound, or a pharmaceutically acceptable salt thereof.

In an aspect, the invention provides a method of inhibiting BRM in a cell of a subject, the method including administering to the subject an effective amount of a compound, N-(1-((4-(6-(2,6-dimethylmorpholino)pyridin-2-yl)thiazol-2-yl)amino)-3-methoxy-1-oxopropan-2-yl)-1-(methylsulfonyl)-1H-pyrrole-3-carboxamide, or a pharmaceutically acceptable salt thereof, at least once daily for six to eight days, followed by six to eight days without administration of the compound, or a pharmaceutically acceptable salt thereof.

In an aspect, the invention provides a method of inhibiting BRM in a cell of a subject, the method including administering to the subject an effective amount of a compound, N-(1-((4-(6-(2,6-dimethylmorpholino)pyridin-2-yl)thiazol-2-yl)amino)-3-methoxy-1-oxopropan-2-yl)-1-(methylsulfonyl)-1H-pyrrole-3-carboxamide, or a pharmaceutically acceptable salt thereof, at least once daily for thirteen to fifteen days, followed by six to eight days without administration of the compound, or a pharmaceutically acceptable salt thereof.

In an aspect, the invention provides a method of inhibiting BRM in a cell of a subject, the method including administering to the subject an effective amount of a compound, N-(1-((4-(6-(2,6-dimethylmorpholino)pyridin-2-yl)thiazol-2-yl)amino)-3-methoxy-1-oxopropan-2-yl)-1-(methylsulfonyl)-1H-pyrrole-3-carboxamide, or a pharmaceutically acceptable salt thereof, at least once daily for six to fifteen days, followed by six to eight days without administration of the compound, or a pharmaceutically acceptable salt thereof.

In an aspect, the invention provides a method of inhibiting BRM in a cell of a subject, the method including administering to the subject an effective amount of a compound, N-(1-((4-(6-(2,6-dimethylmorpholino)pyridin-2-yl)thiazol-2-yl)amino)-3-methoxy-1-oxopropan-2-yl)-1-(methylsulfonyl)-1H-pyrrole-3-carboxamide, or a pharmaceutically acceptable salt thereof, at least once daily for seven days, followed by seven days without administration of the compound, or a pharmaceutically acceptable salt thereof.

In an aspect, the invention provides a method of inhibiting BRM in a cell of a subject, the method including administering to the subject an effective amount of a compound, N-(1-((4-(6-(2,6-dimethylmorpholino)pyridin-2-yl)thiazol-2-yl)amino)-3-methoxy-1-oxopropan-2-yl)-1-(methylsulfonyl)-1H-pyrrole-3-carboxamide, or a pharmaceutically acceptable salt thereof, at least once daily for fourteen days, followed by seven days without administration of the compound, or a pharmaceutically acceptable salt thereof.

In an aspect, the invention provides a method of inhibiting BRM in a cell of a subject, the method including administering to the subject an effective amount of a compound, N-(1-((4-(6-(2,6-dimethylmorpholino)pyridin-2-yl)thiazol-2-yl)amino)-3-methoxy-1-oxopropan-2-yl)-1-(methylsulfonyl)-1H-pyrrole-3-carboxamide, or a pharmaceutically acceptable salt thereof, at least once daily for seven to fourteen days, followed by seven days without administration of the compound, or a pharmaceutically acceptable salt thereof.

In an aspect, the invention provides a method of inhibiting BRG1 in a cell of a subject, the method including administering to the subject an effective amount of a compound, N-(1-((4-(6-(2,6-dimethylmorpholino)pyridin-2-yl)thiazol-2-yl)amino)-3-methoxy-1-oxopropan-2-yl)-1-(methylsulfonyl)-1H-pyrrole-3-carboxamide, or a pharmaceutically acceptable salt thereof, at least once daily for six to eight days, followed by six to eight days without administration of the compound, or a pharmaceutically acceptable salt thereof.

In an aspect, the invention provides a method of inhibiting BRG1 in a cell of a subject, the method including administering to the subject an effective amount of a compound, N-(1-((4-(6-(2,6-dimethylmorpholino)pyridin-2-yl)thiazol-2-yl)amino)-3-methoxy-1-oxopropan-2-yl)-1-(methylsulfonyl)-1H-pyrrole-3-carboxamide, or a pharmaceutically acceptable salt thereof, at least once daily for thirteen to fifteen days, followed by six to eight days without administration of the compound, or a pharmaceutically acceptable salt thereof.

In an aspect, the invention provides a method of inhibiting BRG1 in a cell of a subject, the method including administering to the subject an effective amount of a compound, N-(1-((4-(6-(2,6-dimethylmorpholino)pyridin-2-yl)thiazol-2-yl)amino)-3-methoxy-1-oxopropan-2-yl)-1-(methylsulfonyl)-1H-pyrrole-3-carboxamide, or a pharmaceutically acceptable salt thereof, at least once daily for six to fifteen days, followed by six to eight days without administration of the compound, or a pharmaceutically acceptable salt thereof.

In an aspect, the invention provides a method of inhibiting BRG1 in a cell of a subject, the method including administering to the subject an effective amount of a compound, N-(1-((4-(6-(2,6-dimethylmorpholino)pyridin-2-yl)thiazol-2-yl)amino)-3-methoxy-1-oxopropan-2-yl)-1-(methylsulfonyl)-1H-pyrrole-3-carboxamide, or a pharmaceutically acceptable salt thereof, at least once daily for seven days, followed by seven days without administration of the compound, or a pharmaceutically acceptable salt thereof.

In an aspect, the invention provides a method of inhibiting BRG1 in a cell of a subject, the method including administering to the subject an effective amount of a compound, N-(1-((4-(6-(2,6-dimethylmorpholino)pyridin-2-yl)thiazol-2-yl)amino)-3-methoxy-1-oxopropan-2-yl)-1-(methylsulfonyl)-1H-pyrrole-3-carboxamide, or a pharmaceutically acceptable salt thereof, at least once daily for fourteen days, followed by seven days without administration of the compound, or a pharmaceutically acceptable salt thereof.

In an aspect, the invention provides a method of inhibiting BRG1 in a cell of a subject, the method including administering to the subject an effective amount of a compound, N-(1-((4-(6-(2,6-dimethylmorpholino)pyridin-2-yl)thiazol-2-yl)amino)-3-methoxy-1-oxopropan-2-yl)-1-(methylsulfonyl)-1H-pyrrole-3-carboxamide, or a pharmaceutically acceptable salt thereof, at least once daily for seven to fourteen days, followed by seven days without administration of the compound, or a pharmaceutically acceptable salt thereof.

In an aspect, the invention provides a method of inducing apoptosis in a cell in a subject, the method including administering to the subject an effective amount of a compound, N-(1-((4-(6-(2,6-dimethylmorpholino)pyridin-2-yl)thiazol-2-yl)amino)-3-methoxy-1-oxopropan-2-yl)-1-(methylsulfonyl)-1H-pyrrole-3-carboxamide, or a pharmaceutically acceptable salt thereof, at least once daily for six to eight days, followed by six to eight days without administration of the compound, or a pharmaceutically acceptable salt thereof.

In an aspect, the invention provides a method of inducing apoptosis in a cell in a subject, the method including administering to the subject an effective amount of a compound, N-(1-((4-(6-(2,6-dimethylmorpholino)pyridin-2-yl)thiazol-2-yl)amino)-3-methoxy-1-oxopropan-2-yl)-1-(methylsulfonyl)-1H-pyrrole-3-carboxamide, or a pharmaceutically acceptable salt thereof, at least once daily for thirteen to fifteen days, followed by six to eight days without administration of the compound, or a pharmaceutically acceptable salt thereof.

In an aspect, the invention provides a method of inducing apoptosis in a cell in a subject, the method including administering to the subject an effective amount of a compound, N-(1-((4-(6-(2,6-dimethylmorpholino)pyridin-2-yl)thiazol-2-yl)amino)-3-methoxy-1-oxopropan-2-yl)-1-(methylsulfonyl)-1H-pyrrole-3-carboxamide, or a pharmaceutically acceptable salt thereof, at least once daily for six to fifteen days, followed by six to eight days without administration of the compound, or a pharmaceutically acceptable salt thereof.

In an aspect, the invention provides a method of inducing apoptosis in a cell in a subject, the method including administering to the subject an effective amount of a compound, N-(1-((4-(6-(2,6-dimethylmorpholino)pyridin-2-yl)thiazol-2-yl)amino)-3-methoxy-1-oxopropan-2-yl)-1-(methylsulfonyl)-1H-pyrrole-3-carboxamide, or a pharmaceutically acceptable salt thereof, at least once daily for seven days, followed by seven days without administration of the compound, or a pharmaceutically acceptable salt thereof.

In an aspect, the invention provides a method of inducing apoptosis in a cell in a subject, the method including administering to the subject an effective amount of a compound, N-(1-((4-(6-(2,6-dimethylmorpholino)pyridin-2-yl)thiazol-2-yl)amino)-3-methoxy-1-oxopropan-2-yl)-1-(methylsulfonyl)-1H-pyrrole-3-carboxamide, or a pharmaceutically acceptable salt thereof, at least once daily for fourteen days, followed by seven days without administration of the compound, or a pharmaceutically acceptable salt thereof.

In an aspect, the invention provides a method of inducing apoptosis in a cell in a subject, the method including administering to the subject an effective amount of a compound, N-(1-((4-(6-(2,6-dimethylmorpholino)pyridin-2-yl)thiazol-2-yl)amino)-3-methoxy-1-oxopropan-2-yl)-1-(methylsulfonyl)-1H-pyrrole-3-carboxamide, or a pharmaceutically acceptable salt thereof, at least once daily for seven to fourteen days, followed by seven days without administration of the compound, or a pharmaceutically acceptable salt thereof.

In some embodiments, the cell is a cancer cell and/or the subject has cancer.

In some embodiments, the cancer is metastatic.

In some embodiments, the effective amount is an amount sufficient to reduce cancer tumor growth in the subject compared to a subject that is not administered the compound or a pharmaceutically acceptable salt thereof. In some embodiments, the effective amount is an amount sufficient to suppress metastatic progression of cancer in the subject compared to a subject that is not administered the compound or a pharmaceutically acceptable salt thereof. In some embodiments, the effective amount is an amount sufficient to suppress metastatic colonization of cancer in the subject compared to a subject that is not administered the compound or a pharmaceutically acceptable salt thereof.

In some embodiments, the cancer is non-small cell lung cancer, colorectal cancer, bladder cancer, cancer of unknown primary, glioma, breast cancer, melanoma, non-melanoma skin cancer, endometrial cancer, esophagogastric cancer, pancreatic cancer, hepatobiliary cancer, soft tissue sarcoma, ovarian cancer, head and neck cancer, renal cell carcinoma, bone cancer, non-Hodgkin lymphoma, small-cell lung cancer, prostate cancer, embryonal tumor, germ cell tumor, cervical cancer, thyroid cancer, salivary gland cancer, gastrointestinal neuroendocrine tumor, uterine sarcoma, gastrointestinal stromal tumor, CNS cancer, thymic tumor, adrenocortical carcinoma, appendiceal cancer, small bowel cancer, penile cancer, bone cancer, or hematologic cancer.

In some embodiments, the cancer is non-small cell lung cancer, colorectal cancer, bladder cancer, cancer of unknown primary, glioma, breast cancer, melanoma, non-melanoma skin cancer, endometrial cancer, penile cancer, bone cancer, renal cell carcinoma, prostate cancer, or hematologic cancer.

In some embodiments, the cancer is melanoma, prostate cancer, breast cancer, bone cancer, renal cell carcinoma, or hematologic cancer. In some embodiments, the cancer is melanoma. In some embodiments, the melanoma is uveal melanoma, mucosal melanoma, or cutaneous melanoma. In some embodiments, the melanoma is uveal melanoma.

In some embodiments, the cancer is prostate cancer.

In some embodiments, the cancer is hematologic cancer. In some embodiments, the hematologic cancer is multiple myeloma, large cell lymphoma, acute T-cell leukemia, acute myeloid leukemia, myelodysplastic syndrome, immunoglobulin A lambda myeloma, diffuse mixed histiocytic and lymphocytic lymphoma, B-cell lymphoma, acute lymphoblastic leukemia, diffuse large cell lymphoma, or non-Hodgkin's lymphoma. In some embodiments, the hematologic cancer is acute myeloid leukemia or myelodysplastic syndrome. In some embodiments, the hematologic cancer is relapsed or refractory acute myeloid leukemia.

In some embodiments, the cancer is breast cancer. In some embodiments, the breast cancer is an ER positive breast cancer, an ER negative breast cancer, triple positive breast cancer, or triple negative breast cancer.

In some embodiments, the cancer is a bone cancer. In some embodiments, the bone cancer is Ewing's sarcoma.

In some embodiments, the cancer is a renal cell carcinoma. In some embodiments, the renal cell carcinoma is a microphthalmia transcription factor family translocation renal cell carcinoma.

In some embodiments, the cancer expresses BRG1 and/or BRM protein. In some embodiments, the BRG1 loss of function mutation is in the ATPase catalytic domain of the protein. In some embodiments, the BRG1 loss of function mutation is a deletion at the C-terminus of BRG1.

In some embodiments, the compound or a pharmaceutically acceptable salt thereof is administered in a total dose of between about 5.0 mg and about 25 mg per day. In some embodiments, the compound or a pharmaceutically acceptable salt thereof is administered in a total dose of between about 5.0 mg and about 15 mg per day. In some embodiments, the compound or a pharmaceutically acceptable salt thereof is administered in a total dose of between about 5.0 mg and about 10 mg per day. In some embodiments, the compound or a pharmaceutically acceptable salt thereof is administered in a total dose of between about 5.0 mg and about 7.5 mg per day. In some embodiments, the compound or a pharmaceutically acceptable salt thereof is administered in a total dose of between about 7.5 mg and about 25 mg per day. In some embodiments, the compound or a pharmaceutically acceptable salt thereof is administered in a total dose of between about 7.5 mg and about 15 mg per day. In some embodiments, the compound or a pharmaceutically acceptable salt thereof is administered in a total dose of between about 7.5 mg and about 10 mg per day. In some embodiments, the compound or a pharmaceutically acceptable salt thereof is administered in a total dose of between about 10 mg and about 25 mg per day. In some embodiments, the compound or a pharmaceutically acceptable salt thereof is administered in a total dose of between about 10 mg and about 15 mg per day. In some embodiments, the compound or a pharmaceutically acceptable salt thereof is administered in a total dose of between about 15 mg and about 25 mg per day. In some embodiments, the compound is administered in a total dose of about 5.0 mg per day. In some embodiments, the compound is administered in a total dose of about 7.5 mg per day. In some embodiments, the compound is administered in a total dose of about 10 mg per day. In some embodiments, the compound is administered in a total dose of about 17.5 mg per day. In some embodiments, the compound is administered in a total dose of about 15 mg per day. In some embodiments, the compound is administered in a total dose of about 20 mg per day. In some embodiments, the compound is administered in a total dose of about 22.5 mg per day.

In some embodiments, the total dose is administered to the subject once per day.

In some embodiments, the method includes at least 21 days of treatment. In some embodiments, the method includes at least 28 days of treatment.

In some embodiments, the compound or a pharmaceutically acceptable salt thereof is administered daily for six to fifteen days immediately following the period of six to eight days without administration of the compound or a pharmaceutically acceptable salt thereof.

In some embodiments, the compound or pharmaceutically acceptable salt thereof, is administered orally.

In some embodiments of any of the aspects described herein, the compound is:

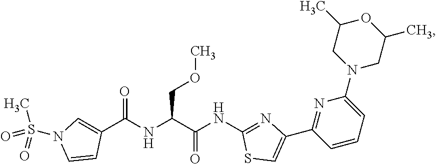 [SMILES: COC[C@H](NC(=O)c1ccn(S(C)(=O)=O)c1)C(=O)Nc1nc(-c2cccc(N3CC(C)OC(C)C3)n2)cs1]

or a pharmaceutically acceptable salt thereof.

In some embodiments of any of the aspects described herein, the compound is:

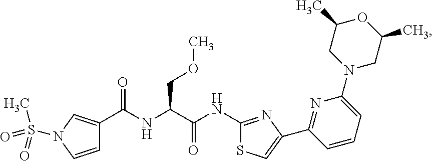 [SMILES: COC[C@H](NC(=O)c1ccn(S(C)(=O)=O)c1)C(=O)Nc1nc(-c2cccc(N3C[C@@H](C)O[C@@H](C)C3)n2)cs1]

or a pharmaceutically acceptable salt thereof.

In some embodiments of any of the aspects described herein, the compound is

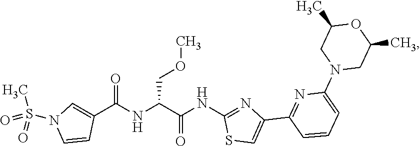 [SMILES: COC[C@@H](NC(=O)c1ccn(S(C)(=O)=O)c1)C(=O)Nc1nc(-c2cccc(N3C[C@@H](C)O[C@@H](C)C3)n2)cs1]

or a pharmaceutically acceptable salt thereof.

### Chemical Terms

Compounds of the disclosure can have one or more asymmetric carbon atoms and can exist in the form of optically pure enantiomers, mixtures of enantiomers such as, for example, racemates, optically pure diastereoisomers, mixtures of diastereoisomers, diastereoisomeric racemates, or mixtures of diastereoisomeric racemates. The optically active forms can be obtained for example by resolution of the racemates, by asymmetric synthesis or asymmetric chromatography (chromatography with a chiral adsorbent or eluant). That is, certain of the disclosed compounds may exist in various stereoisomeric forms. Stereoisomers are compounds that differ only in their spatial arrangement. Enantiomers are pairs of stereoisomers whose mirror images are not superimposable, most commonly because they contain an asymmetrically substituted carbon atom that acts as a chiral center. “Enantiomer” means one of a pair of molecules that are mirror images of each other and are not superimposable. Diastereomers are stereoisomers that are not related as mirror images, most commonly because they contain two or more asymmetrically substituted carbon atoms and represent the configuration of substituents around one or more chiral carbon atoms. Enantiomers of a compound can be prepared, for example, by separating an enantiomer from a racemate using one or more well-known techniques and methods, such as, for example, chiral chromatography and separation methods based thereon. The appropriate technique and/or method for separating an enantiomer of a compound described herein from a racemic mixture can be readily determined by those of skill in the art. “Racemate” or “racemic mixture” means a compound containing two enantiomers, wherein such mixtures exhibit no optical activity; i.e., they do not rotate the plane of polarized light. “Geometric isomer” means isomers that differ in the orientation of substituent atoms in relationship to a carbon-carbon double bond, to a cycloalkyl ring, or to a bridged bicyclic system. Atoms (other than H) on each side of a carbon-carbon double bond may be in an E (substituents are on opposite sides of the carbon-carbon double bond) or Z (substituents are oriented on the same side) configuration. “R,” “S,” “S*,” “R*,” “E,” “Z,” “cis,” and “trans,” indicate configurations relative to the core molecule. Certain of the disclosed compounds may exist in atropisomeric forms. Atropisomers are stereoisomers resulting from hindered rotation about single bonds where the steric strain barrier to rotation is high enough to allow for the isolation of the conformers. The compounds of the disclosure may be prepared as individual isomers by either isomer-specific synthesis or resolved from an isomeric mixture. Conventional resolution techniques include forming the salt of a free base of each isomer of an isomeric pair using an optically active acid (followed by fractional crystallization and regeneration of the free base), forming the salt of the acid form of each isomer of an isomeric pair using an optically active amine (followed by fractional crystallization and regeneration of the free acid), forming an ester or amide of each of the isomers of an isomeric pair using an optically pure acid, amine or alcohol (followed by chromatographic separation and removal of the chiral auxiliary), or resolving an isomeric mixture of either a starting material or a final product using various well known chromatographic methods. When the stereochemistry of a disclosed compound is named or depicted by structure, the named or depicted stereoisomer is at least 60%, 70%, 80%, 90%, 99%, or 99.9% by weight relative to the other stereoisomers. When a single enantiomer is named or depicted by structure, the depicted or named enantiomer is at least 60%, 70%, 80%, 90%, 99%, or 99.9% by weight optically pure. When a single diastereomer is named or depicted by structure, the depicted or named diastereomer is at least 60%, 70%, 80%, 90%, 99%, or 99.9% by weight pure. Percent optical purity is the ratio of the weight of the enantiomer or over the weight of the enantiomer plus the weight of its optical isomer. Diastereomeric purity by weight is the ratio of the weight of one diastereomer or over the weight of all the diastereomers. When the stereochemistry of a disclosed compound is named or depicted by structure, the named or depicted stereoisomer is at least 60%, 70%, 80%, 90%, 99%, or 99.9% by mole fraction pure relative to the other stereoisomers. When a single enantiomer is named or depicted by structure, the depicted or named enantiomer is at least 60%, 70%, 80%, 90%, 99%, or 99.9% by mole fraction pure. When a single diastereomer is named or depicted by structure, the depicted or named diastereomer is at least 60%, 70%, 80%, 90%, 99%, or 99.9% by mole fraction pure. Percent purity by mole fraction is the ratio of the moles of the enantiomer or over the moles of the enantiomer plus the moles of its optical isomer. Similarly, percent purity by moles fraction is the ratio of the moles of the diastereomer or over the moles of the diastereomer plus the moles of its isomer. When a disclosed compound is named or depicted by structure without indicating the stereochemistry, and the compound has at least one chiral center, it is to be understood that the name or structure encompasses either enantiomer of the compound free from the corresponding optical isomer, a racemic mixture of the compound, or mixtures enriched in one enantiomer relative to its corresponding optical isomer. When a disclosed compound is named or depicted by structure without indicating the stereochemistry and has two or more chiral centers, it is to be understood that the name or structure encompasses a diastereomer free of other diastereomers, a number of diastereomers free from other diastereomeric pairs, mixtures of diastereomers, mixtures of diastereomeric pairs, mixtures of diastereomers in which one diastereomer is enriched relative to the other diastereomer(s), or mixtures of diastereomers in which one or more diastereomer is enriched relative to the other diastereomers. The invention embraces all of these forms.

Unless otherwise defined, all technical and scientific terms used herein have the same meaning as commonly understood by one of ordinary skill in the art to which this disclosure belongs. Methods and materials are described herein for use in the present disclosure; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

### Definitions

In this application, unless otherwise clear from context, (i) the term “a” may be understood to mean “at least one”; (ii) the term “or” may be understood to mean “and/or”; and (iii) the terms “comprising” and “including” may be understood to encompass itemized components or steps whether presented by themselves or together with one or more additional components or steps.

As used herein, the terms “about” and “approximately” refer to a value that is within 10% above or below the value being described. For example, the term “about 5 nM” indicates a range of from 4.5 to 5.5 nM.

As used herein, the term “administration” refers to the administration of a composition (e.g., a compound or a preparation that includes a compound as described herein) to a subject or system. Administration to an animal subject (e.g., to a human) may be by any appropriate route. For example, in some embodiments, administration may be bronchial (including by bronchial instillation), buccal, enteral, interdermal, intra-arterial, intradermal, intragastric, intramedullary, intramuscular, intranasal, intraperitoneal, intrathecal, intratumoral, intravenous, intraventricular, mucosal, nasal, oral, rectal, subcutaneous, sublingual, topical, tracheal (including by intratracheal instillation), transdermal, vaginal, and vitreal.

As used herein, the term “BAF complex” refers to the BRG1- or HBRM-associated factors complex in a human cell.

As used herein, the term “BAF complex-related disorder” refers to a disorder that is caused or affected by the level of activity of a BAF complex.

As used herein, the term “BRG1” refers to ATP-dependent chromatin remodeler SMARCA4. BRG1 is a component of the BAF complex, a SWI/SNF ATPase chromatin remodeling complex. Human BRG1 is encoded by the SMARCA4 gene on chromosome 19, a nucleic acid sequence of which is set forth in SEQ ID NO: 1 (GenBank Accession No.: NM_001128849.1 (mRNA);

www.ncbi.nlm.nih.gov/nuccore/NM_001128849.1?report=fasta).

GGCGGGGGAGGCGCCGGGAAGTCGACGGCGCCGGCGGCTCCTGCAGGAGGCCACTGTCTGCAGCTCCCGT
GAAGATGTCCACTCCAGACCCACCCCTGGGCGGAACTCCTCGGCCAGGTCCTTCCCCGGGCCCTGGCCCT
TCCCCTGGAGCCATGCTGGGCCCTAGCCCGGGTCCCTCGCCGGGCTCCGCCCACAGCATGATGGGGCCCA
GCCCAGGGCCGCCCTCAGCAGGACACCCCATCCCCACCCAGGGGCCTGGAGGGTACCCTCAGGACAACAT
GCACCAGATGCACAAGCCCATGGAGTCCATGCATGAGAAGGGCATGTCGGACGACCCGCGCTACAACCAG
ATGAAAGGAATGGGGATGCGGTCAGGGGGCCATGCTGGGATGGGGCCCCCGCCCAGCCCCATGGACCAGC
ACTCCCAAGGTTACCCCTCGCCCCTGGGTGGCTCTGAGCATGCCTCTAGTCCAGTTCCAGCCAGTGGCCC
GTCTTCGGGGCCCCAGATGTCTTCCGGGCCAGGAGGTGCCCCGCTGGATGGTGCTGACCCCCAGGCCTTG
GGGCAGCAGAACCGGGGCCCAACCCCATTTAACCAGAACCAGCTGCACCAGCTCAGAGCTCAGATCATGG
CCTACAAGATGCTGGCCAGGGGGCAGCCCCTCCCCGACCACCTGCAGATGGCGGTGCAGGGCAAGCGGCC
GATGCCCGGGATGCAGCAGCAGATGCCAACGCTACCTCCACCCTCGGTGTCCGCAACAGGACCCGGCCCT
GGCCCTGGCCCTGGCCCCGGCCCGGGTCCCGGCCCGGCACCTCCAAATTACAGCAGGCCTCATGGTATGG
GAGGGCCCAACATGCCTCCCCCAGGACCCTCGGGCGTGCCCCCCGGGATGCCAGGCCAGCCTCCTGGAGG
GCCTCCCAAGCCCTGGCCTGAAGGACCCATGGCGAATGCTGCTGCCCCCACGAGCACCCCTCAGAAGCTG
ATTCCCCCGCAGCCAACGGGCCGCCCTTCCCCCGCGCCCCCTGCCGTCCCACCCGCCGCCTCGCCCGTGA
TGCCACCGCAGACCCAGTCCCCCGGGCAGCCGGCCCAGCCCGCGCCCATGGTGCCACTGCACCAGAAGCA
GAGCCGCATCACCCCCATCCAGAAGCCGCGGGGCCTCGACCCTGTGGAGATCCTGCAGGAGCGCGAGTAC
AGGCTGCAGGCTCGCATCGCACACCGAATTCAGGAACTTGAAAACCTTCCCGGGTCCCTGGCCGGGGATT
TGCGAACCAAAGCGACCATTGAGCTCAAGGCCCTCAGGCTGCTGAACTTCCAGAGGCAGCTGCGCCAGGA
GGTGGTGGTGTGCATGCGGAGGGACACAGCGCTGGAGACAGCCCTCAATGCTAAGGCCTACAAGCGCAGC
AAGCGCCAGTCCCTGCGCGAGGCCCGCATCACTGAGAAGCTGGAGAAGCAGCAGAAGATCGAGCAGGAGC
GCAAGCGCCGGCAGAAGCACCAGGAATACCTCAATAGCATTCTCCAGCATGCCAAGGATTTCAAGGAATA
TCACAGATCCGTCACAGGCAAAATCCAGAAGCTGACCAAGGCAGTGGCCACGTACCATGCCAACACGGAG
CGGGAGCAGAAGAAAGAGAACGAGCGGATCGAGAAGGAGCGCATGCGGAGGCTCATGGCTGAAGATGAGG
AGGGGTACCGCAAGCTCATCGACCAGAAGAAGGACAAGCGCCTGGCCTACCTCTTGCAGCAGACAGACGA
GTACGTGGCTAACCTCACGGAGCTGGTGCGGCAGCACAAGGCTGCCCAGGTCGCCAAGGAGAAAAAGAAG
AAAAAGAAAAAGAAGAAGGCAGAAAATGCAGAAGGACAGACGCCTGCCATTGGGCCGGATGGCGAGCCTC
TGGACGAGACCAGCCAGATGAGCGACCTCCCGGTGAAGGTGATCCACGTGGAGAGTGGGAAGATCCTCAC
AGGCACAGATGCCCCCAAAGCCGGGCAGCTGGAGGCCTGGCTCGAGATGAACCCGGGGTATGAAGTAGCT
CCGAGGTCTGATAGTGAAGAAAGTGGCTCAGAAGAAGAGGAAGAGGAGGAGGAGGAAGAGCAGCCGCAGG
CAGCACAGCCTCCCACCCTGCCCGTGGAGGAGAAGAAGAAGATTCCAGATCCAGACAGCGATGACGTCTC
TGAGGTGGACGCGCGGCACATCATTGAGAATGCCAAGCAAGATGTCGATGATGAATATGGCGTGTCCCAG
GCCCTTGCACGTGGCCTGCAGTCCTACTATGCCGTGGCCCATGCTGTCACTGAGAGAGTGGACAAGCAGT
CAGCGCTTATGGTCAATGGTGTCCTCAAACAGTACCAGATCAAAGGTTTGGAGTGGCTGGTGTCCCTGTA
CAACAACAACCTGAACGGCATCCTGGCCGACGAGATGGGCCTGGGGAAGACCATCCAGACCATCGCGCTC
ATCACGTACCTCATGGAGCACAAACGCATCAATGGGCCCTTCCTCATCATCGTGCCTCTCTCAACGCTGT
CCAACTGGGCGTACGAGTTTGACAAGTGGGCCCCCTCCGTGGTGAAGGTGTCTTACAAGGGATCCCCAGC
AGCAAGACGGGCCTTTGTCCCCCAGCTCCGGAGTGGGAAGTTCAACGTCTTGCTGACGACGTACGAGTAC
ATCATCAAAGACAAGCACATCCTCGCCAAGATCCGTTGGAAGTACATGATTGTGGACGAAGGTCACCGCA
TGAAGAACCACCACTGCAAGCTGACGCAGGTGCTCAACACGCACTATGTGGCACCCCGCCGCCTGCTGCT
GACGGGCACACCGCTGCAGAACAAGCTTCCCGAGCTCTGGGCGCTGCTCAACTTCCTGCTGCCCACCATC
TTCAAGAGCTGCAGCACCTTCGAGCAGTGGTTTAACGCACCCTTTGCCATGACCGGGGAAAAGGTGGACC
TGAATGAGGAGGAAACCATTCTCATCATCCGGCGTCTCCACAAAGTGCTGCGGCCCTTCTTGCTCCGACG
ACTCAAGAAGGAAGTCGAGGCCCAGTTGCCCGAAAAGGTGGAGTACGTCATCAAGTGCGACATGTCTGCG
CTGCAGCGAGTGCTCTACCGCCACATGCAGGCCAAGGGCGTGCTGCTGACTGATGGCTCCGAGAAGGACA
AGAAGGGCAAAGGCGGCACCAAGACCCTGATGAACACCATCATGCAGCTGCGGAAGATCTGCAACCACCC
CTACATGTTCCAGCACATCGAGGAGTCCTTTTCCGAGCACTTGGGGTTCACTGGCGGCATTGTCCAAGGG
CTGGACCTGTACCGAGCCTCGGGTAAATTTGAGCTTCTTGATAGAATTCTTCCCAAACTCCGAGCAACCA
ACCACAAAGTGCTGCTGTTCTGCCAAATGACCTCCCTCATGACCATCATGGAAGATTACTTTGCGTATCG
CGGCTTTAAATACCTCAGGCTTGATGGAACCACGAAGGCGGAGGACCGGGGCATGCTGCTGAAAACCTTC
AACGAGCCCGGCTCTGAGTACTTCATCTTCCTGCTCAGCACCCGGGCTGGGGGGCTCGGCCTGAACCTCC
AGTCGGCAGACACTGTGATCATTTTTGACAGCGACTGGAATCCTCACCAGGACCTGCAAGCGCAGGACCG
AGCCCACCGCATCGGGCAGCAGAACGAGGTGCGTGTGCTCCGCCTCTGCACCGTCAACAGCGTGGAGGAG
AAGATCCTAGCTGCAGCCAAGTACAAGCTCAACGTGGACCAGAAGGTGATCCAGGCCGGCATGTTCGACC
AGAAGTCCTCCAGCCATGAGCGGCGCGCCTTCCTGCAGGCCATCCTGGAGCACGAGGAGCAGGATGAGAG
CAGACACTGCAGCACGGGCAGCGGCAGTGCCAGCTTCGCCCACACTGCCCCTCCGCCAGCGGGCGTCAAC
CCCGACTTGGAGGAGCCACCTCTAAAGGAGGAAGACGAGGTGCCCGACGACGAGACCGTCAACCAGATGA
TCGCCCGGCACGAGGAGGAGTTTGATCTGTTCATGCGCATGGACCTGGACCGCAGGCGCGAGGAGGCCCG
CAACCCCAAGCGGAAGCCGCGCCTCATGGAGGAGGACGAGCTCCCCTCGTGGATCATCAAGGACGACGCG
GAGGTGGAGCGGCTGACCTGTGAGGAGGAGGAGGAGAAGATGTTCGGCCGTGGCTCCCGCCACCGCAAGG
AGGTGGACTACAGCGACTCACTGACGGAGAAGCAGTGGCTCAAGAAAATTACAGGAAAAGATATCCATGA
CACAGCCAGCAGTGTGGCACGTGGGCTACAATTCCAGCGTGGCCTTCAGTTCTGCACACGTGCGTCAAAG
GCCATCGAGGAGGGCACGCTGGAGGAGATCGAAGAGGAGGTCCGGCAGAAGAAATCATCACGGAAGCGCA
AGCGAGACAGCGACGCCGGCTCCTCCACCCCGACCACCAGCACCCGCAGCCGCGACAAGGACGACGAGAG
CAAGAAGCAGAAGAAGCGCGGGCGGCCGCCTGCCGAGAAACTCTCCCCTAACCCACCCAACCTCACCAAG
AAGATGAAGAAGATTGTGGATGCCGTGATCAAGTACAAGGACAGCAGCAGTGGACGTCAGCTCAGCGAGG
TCTTCATCCAGCTGCCCTCGCGAAAGGAGCTGCCCGAGTACTACGAGCTCATCCGCAAGCCCGTGGACTT
CAAGAAGATAAAGGAGCGCATTCGCAACCACAAGTACCGCAGCCTCAACGACCTAGAGAAGGACGTCATG
CTCCTGTGCCAGAACGCACAGACCTTCAACCTGGAGGGCTCCCTGATCTATGAAGACTCCATCGTCTTGC
AGTCGGTCTTCACCAGCGTGCGGCAGAAAATCGAGAAGGAGGATGACAGTGAAGGCGAGGAGAGTGAGGA
GGAGGAAGAGGGCGAGGAGGAAGGCTCCGAATCCGAATCTCGGTCCGTCAAAGTGAAGATCAAGCTTGGC
CGGAAGGAGAAGGCACAGGACCGGCTGAAGGGCGGCCGGCGGCGGCCGAGCCGAGGGTCCCGAGCCAAGC
CGGTCGTGAGTGACGATGACAGTGAGGAGGAACAAGAGGAGGACCGCTCAGGAAGTGGCAGCGAAGAAGA
CTGAGCCCCGACATTCCAGTCTCGACCCCGAGCCCCTCGTTCCAGAGCTGAGATGGCATAGGCCTTAGCA
GTAACGGGTAGCAGCAGATGTAGTTTCAGACTTGGAGTAAAACTGTATAAACAAAAGAATCTTCCATATT
TATACAGCAGAGAAGCTGTAGGACTGTTTGTGACTGGCCCTGTCCTGGCATCAGTAGCATCTGTAACAGC
ATTAACTGTCTTAAAGAGAGAGAGAGAGAATTCCGAATTGGGGAACACACGATACCTGTTTTTCTTTTCC
GTTGCTGGCAGTACTGTTGCGCCGCAGTTTGGAGTCACTGTAGTTAAGTGTGGATGCATGTGCGTCACCG
TCCACTCCTCCTACTGTATTTTATTGGACAGGTCAGACTCGCCGGGGGCCCGGCGAGGGTATGTCAGTGT
CACTGGATGTCAAACAGTAATAAATTAAACCAACAACAAAACGCACAGCCAAAAAAAAA

The term “BRG1” also refers to natural variants of the wild-type human BRG1 protein, such as 50 proteins having at least 85% identity (e.g., 85%, 86%, 87%, 88%, 89%, 90%, 91%, 92%, 93%, 94%, 95%, 96%, 97%, 98%, 99%, 99.9% identity, or more) to an amino acid sequence of wild-type BRG1, which is set forth in SEQ ID NO: 2 (UniProt Accession No.: P51532; www.uniprot.org/uniprot/P51532.fasta).

SEQ ID NO: 2
MSTPDPPLGGTPRPGPSPGPGPSPGAMLGPSPGPSPGSAHSMMGPSPGPP
SAGHPIPTQGPGGYPQDNMHQMHKPMESMHEKGMSDDPRYNQMKGMGMRS
GGHAGMGPPPSPMDQHSQGYPSPLGGSEHASSPVPASGPSSGPQMSSGPG
GAPLDGADPQALGQQNRGPTPFNQNQLHQLRAQIMAYKMLARGQPLPDHL
QMAVQGKRPMPGMQQQMPTLPPPSVSATGPGPGPGPGPGPGPGPAPPNYS
RPHGMGGPNMPPPGPSGVPPGMPGQPPGGPPKPWPEGPMANAAAPTSTPQ
KLIPPQPTGRPSPAPPAVPPAASPVMPPQTQSPGQPAQPAPMVPLHQKQS
RITPIQKPRGLDPVEILQEREYRLQARIAHRIQELENLPGSLAGDLRTKA
TIELKALRLLNFQRQLRQEVVVCMRRDTALETALNAKAYKRSKRQSLREA
RITEKLEKQQKIEQERKRRQKHQEYLNSILQHAKDFKEYHRSVTGKIQKL
TKAVATYHANTEREQKKENERIEKERMRRLMAEDEEGYRKLIDQKKDKRL
AYLLQQTDEYVANLTELVRQHKAAQVAKEKKKKKKKKKAENAEGQTPAIG
PDGEPLDETSQMSDLPVKVIHVESGKILTGTDAPKAGQLEAWLEMNPGYE
VAPRSDSEESGSEEEEEEEEEEQPQAAQPPTLPVEEKKKIPDPDSDDVSE
VDARHIIENAKQDVDDEYGVSQALARGLQSYYAVAHAVTERVDKQSALMV
NGVLKQYQIKGLEWLVSLYNNNLNGILADEMGLGKTIQTIALITYLMEHK
RINGPFLIIVPLSTLSNWAYEFDKWAPSVVKVSYKGSPAARRAFVPQLRS
GKFNVLLTTYEYIIKDKHILAKIRWKYMIVDEGHRMKNHHCKLTQVLNTH
YVAPRRLLLTGTPLQNKLPELWALLNFLLPTIFKSCSTFEQWFNAPFAMT
GEKVDLNEEETILIIRRLHKVLRPFLLRRLKKEVEAQLPEKVEYVIKCDM
SALQRVLYRHMQAKGVLLTDGSEKDKKGKGGTKTLMNTIMQLRKICNHPY
MFQHIEESFSEHLGFTGGIVQGLDLYRASGKFELLDRILPKLRATNHKVL
LFCQMTSLMTIMEDYFAYRGFKYLRLDGTTKAEDRGMLLKTFNEPGSEYF
IFLLSTRAGGLGLNLQSADTVIIFDSDWNPHQDLQAQDRAHRIGQQNEVR
VLRLCTVNSVEEKILAAAKYKLNVDQKVIQAGMEDQKSSSHERRAFLQAI
LEHEEQDESRHCSTGSGSASFAHTAPPPAGVNPDLEEPPLKEEDEVPDDE
TVNQMIARHEEEFDLFMRMDLDRRREEARNPKRKPRLMEEDELPSWIIKD
DAEVERLTCEEEEEKMFGRGSRHRKEVDYSDSLTEKQWLKAIEEGTLEEI
EEEVRQKKSSRKRKRDSDAGSSTPTTSTRSRDKDDESKKQKKRGRPPAEK
LSPNPPNLTKKMKKIVDAVIKYKDSSSGRQLSEVFIQLPSRKELPEYYEL
IRKPVDFKKIKERIRNHKYRSLNDLEKDVMLLCQNAQTFNLEGSLIYEDS
IVLQSVFTSVRQKIEKEDDSEGEESEEEEEGEEEGSESESRSVKVKIKLG
RKEKAQDRLKGGRRRPSRGSRAKPVVSDDDSEEEQEEDRSGSGSEED.

As used herein, the term “BRG1 activity” refers to the BRG1 enzyme ATPase activity.

As used herein, the term “BRG1 loss of function mutation” refers to a mutation in BRG1 that leads to the protein having diminished activity (e.g., at least 1% reduction in BRG1 activity, for example 2%, 5%, 10%, 25%, 50%, or 100% reduction in BRG1 activity). Exemplary BRG1 loss of function mutations include, but are not limited to, a homozygous BRG1 mutation and a deletion at the C-terminus of BRG1.

As used herein, the term “BRG1 loss of function disorder” refers to a disorder (e.g., cancer) that exhibits a reduction in BRG1 activity (e.g., at least 1% reduction in BRG1 activity, for example 2%, 5%, 10%, 25%, 50%, or 100% reduction in BRG1 activity).

As used herein, the term “BRM” refers to probable global transcription activator SNF2L2. BRM is a component of the BAF complex, a SWI/SNF ATPase chromatin remodeling complex. Human BRM is encoded by the SMARCA2 gene on chromosome 9, a nucleic acid sequence of which is set forth in SEQ ID NO: 3 (GenBank Accession No.: NM_003070.4

www.ncbi.nlm.nih.gov/nuccore/NM_003070.4?report=fasta).

SEQ ID NO: 3
GCGTCTTCCGGCGCCCGCGGAGGAGGCGAGGGTGGGACGCTGGGCGGAGCCCGAGTTTAGGAAGAGGAGG
GGACGGCTGTCATCAATGAAGTCATATTCATAATCTAGTCCTCTCTCCCTCTGTTTCTGTACTCTGGGTG
ACTCAGAGAGGGAAGAGATTCAGCCAGCACACTCCTCGCGAGCAAGCATTACTCTACTGACTGGCAGAGA
CAGGAGAGGTAGATGTCCACGCCCACAGACCCTGGTGCGATGCCCCACCCAGGGCCTTCGCCGGGGCCTG
GGCCTTCCCCTGGGCCAATTCTTGGGCCTAGTCCAGGACCAGGACCATCCCCAGGTTCCGTCCACAGCAT
GATGGGGCCAAGTCCTGGACCTCCAAGTGTCTCCCATCCTATGCCGACGATGGGGTCCACAGACTTCCCA
CAGGAAGGCATGCATCAAATGCATAAGCCCATCGATGGTATACATGACAAGGGGATTGTAGAAGACATCC
ATTGTGGATCCATGAAGGGCACTGGTATGCGACCACCTCACCCAGGCATGGGCCCTCCCCAGAGTCCAAT
GGATCAACACAGCCAAGGTTATATGTCACCACACCCATCTCCATTAGGAGCCCCAGAGCACGTCTCCAGC
CCTATGTCTGGAGGAGGCCCAACTCCACCTCAGATGCCACCAAGCCAGCCGGGGGCCCTCATCCCAGGTG
ATCCGCAGGCCATGAGCCAGCCCAACAGAGGTCCCTCACCTTTCAGTCCTGTCCAGCTGCATCAGCTTCG
AGCTCAGATTTTAGCTTATAAAATGCTGGCCCGAGGCCAGCCCCTCCCCGAAACGCTGCAGCTTGCAGTC
CAGGGGAAAAGGACGTTGCCTGGCTTGCAGCAACAACAGCAGCAGCAACAGCAGCAGCAGCAGCAGCAGC
AGCAGCAGCAGCAGCAGCAACAGCAGCCGCAGCAGCAGCCGCCGCAACCACAGACGCAGCAACAACAGCA
GCCGGCCCTTGTTAACTACAACAGACCATCTGGCCCGGGGCCGGAGCTGAGCGGCCCGAGCACCCCGCAG
AAGCTGCCGGTGCCCGCGCCCGGCGGCCGGCCCTCGCCCGCGCCCCCCGCAGCCGCGCAGCCGCCCGCGG
CCGCAGTGCCCGGGCCCTCAGTGCCGCAGCCGGCCCCGGGGCAGCCCTCGCCCGTCCTCCAGCTGCAGCA
GAAGCAGAGCCGCATCAGCCCCATCCAGAAACCGCAAGGCCTGGACCCCGTGGAAATTCTGCAAGAGCGG
GAATACAGACTTCAGGCCCGCATAGCTCATAGGATACAAGAACTGGAAAATCTGCCTGGCTCTTTGCCAC
CAGATTTAAGAACCAAAGCAACCGTGGAACTAAAAGCACTTCGGTTACTCAATTTCCAGCGTCAGCTGAG
ACAGGAGGTGGTGGCCTGCATGCGCAGGGACACGACCCTGGAGACGGCTCTCAACTCCAAAGCATACAAA
CGGAGCAAGCGCCAGACTCTGAGAGAAGCTCGCATGACCGAGAAGCTGGAGAAGCAGCAGAAGATTGAGC
AGGAGAGGAAACGCCGTCAGAAACACCAGGAATACCTGAACAGTATTTTGCAACATGCAAAAGATTTTAA
GGAATATCATCGGTCTGTGGCCGGAAAGATCCAGAAGCTCTCCAAAGCAGTGGCAACTTGGCATGCCAAC
ACTGAAAGAGAGCAGAAGAAGGAGACAGAGCGGATTGAAAAGGAGAGAATGCGGCGACTGATGGCTGAAG
ATGAGGAGGGTTATAGAAAACTGATTGATCAAAAGAAAGACAGGCGTTTAGCTTACCTTTTGCAGCAGAC
CGATGAGTATGTAGCCAATCTGACCAATCTGGTTTGGGAGCACAAGCAAGCCCAGGCAGCCAAAGAGAAG
AAGAAGAGGAGGAGGAGGAAGAAGAAGGCTGAGGAGAATGCAGAGGGTGGGGAGTCTGCCCTGGGACCGG
ATGGAGAGCCCATAGATGAGAGCAGCCAGATGAGTGACCTCCCTGTCAAAGTGACTCACACAGAAACCGG
CAAGGTTCTGTTCGGACCAGAAGCACCCAAAGCAAGTCAGCTGGACGCCTGGCTGGAAATGAATCCTGGT
TATGAAGTTGCCCCTAGATCTGACAGTGAAGAGAGTGATTCTGATTATGAGGAAGAGGATGAGGAAGAAG
AGTCCAGTAGGCAGGAAACCGAAGAGAAAATACTCCTGGATCCAAATAGCGAAGAAGTTTCTGAGAAGGA
TGCTAAGCAGATCATTGAGACAGCTAAGCAAGACGTGGATGATGAATACAGCATGCAGTACAGTGCCAGG
GGCTCCCAGTCCTACTACACCGTGGCTCATGCCATCTCGGAGAGGGTGGAGAAACAGTCTGCCCTCCTAA
TTAATGGGACCCTAAAGCATTACCAGCTCCAGGGCCTGGAATGGATGGTTTCCCTGTATAATAACAACTT
GAACGGAATCTTAGCCGATGAAATGGGGCTTGGAAAGACCATACAGACCATTGCACTCATCACTTATCTG
ATGGAGCACAAAAGACTCAATGGCCCCTATCTCATCATTGTTCCCCTTTCGACTCTATCTAACTGGACAT
ATGAATTTGACAAATGGGCTCCTTCTGTGGTGAAGATTTCTTACAAGGGTACTCCTGCCATGCGTCGCTC
CCTTGTCCCCCAGCTACGGAGTGGCAAATTCAATGTCCTCTTGACTACTTATGAGTATATTATAAAAGAC
AAGCACATTCTTGCAAAGATTCGGTGGAAATACATGATAGTGGACGAAGGCCACCGAATGAAGAATCACC
ACTGCAAGCTGACTCAGGTCTTGAACACTCACTATGTGGCCCCCAGAAGGATCCTCTTGACTGGGACCCC
GCTGCAGAATAAGCTCCCTGAACTCTGGGCCCTCCTCAACTTCCTCCTCCCAACAATTTTTAAGAGCTGC
AGCACATTTGAACAATGGTTCAATGCTCCATTTGCCATGACTGGTGAAAGGGTGGACTTAAATGAAGAAG
AAACTATATTGATCATCAGGCGTCTACATAAGGTGTTAAGACCATTTTTACTAAGGAGACTGAAGAAAGA
AGTTGAATCCCAGCTTCCCGAAAAAGTGGAATATGTGATCAAGTGTGACATGTCAGCTCTGCAGAAGATT
CTGTATCGCCATATGCAAGCCAAGGGGATCCTTCTCACAGATGGTTCTGAGAAAGATAAGAAGGGGAAAG
GAGGTGCTAAGACACTTATGAACACTATTATGCAGTTGAGAAAAATCTGCAACCACCCATATATGTTTCA
GCACATTGAGGAATCCTTTGCTGAACACCTAGGCTATTCAAATGGGGTCATCAATGGGGCTGAACTGTAT
CGGGCCTCAGGGAAGTTTGAGCTGCTTGATCGTATTCTGCCAAAATTGAGAGCGACTAATCACCGAGTGC
TGCTTTTCTGCCAGATGACATCTCTCATGACCATCATGGAGGATTATTTTGCTTTTCGGAACTTCCTTTA
CCTACGCCTTGATGGCACCACCAAGTCTGAAGATCGTGCTGCTTTGCTGAAGAAATTCAATGAACCTGGA
TCCCAGTATTTCATTTTCTTGCTGAGCACAAGAGCTGGTGGCCTGGGCTTAAATCTTCAGGCAGCTGATA
CAGTGGTCATCTTTGACAGCGACTGGAATCCTCATCAGGATCTGCAGGCCCAAGACCGAGCTCACCGCAT
CGGGCAGCAGAACGAGGTCCGGGTACTGAGGCTCTGTACCGTGAACAGCGTGGAGGAAAAGATCCTCGCG
GCCGCAAAATACAAGCTGAACGTGGATCAGAAAGTGATCCAGGCGGGCATGTTTGACCAAAAGTCTTCAA
GCCACGAGCGGAGGGCATTCCTGCAGGCCATCTTGGAGCATGAGGAGGAAAATGAGGAAGAAGATGAAGT
ACCGGACGATGAGACTCTGAACCAAATGATTGCTCGACGAGAAGAAGAATTTGACCTTTTTATGCGGATG
GACATGGACCGGCGGAGGGAAGATGCCCGGAACCCGAAACGGAAGCCCCGTTTAATGGAGGAGGATGAGC
TGCCCTCCTGGATCATTAAGGATGACGCTGAAGTAGAAAGGCTCACCTGTGAAGAAGAGGAGGAGAAAAT
ATTTGGGAGGGGGTCCCGCCAGCGCCGTGACGTGGACTACAGTGACGCCCTCACGGAGAAGCAGTGGCTA
AGGGCCATCGAAGACGGCAATTTGGAGGAAATGGAAGAGGAAGTACGGCTTAAGAAGCGAAAAAGACGAA
GAAATGTGGATAAAGATCCTGCAAAAGAAGATGTGGAAAAAGCTAAGAAGAGAAGAGGCCGCCCTCCCGC
TGAGAAACTGTCACCAAATCCCCCCAAACTGACAAAGCAGATGAACGCTATCATCGATACTGTGATAAAC
TACAAAGATAGGTGTAACGTGGAGAAGGTGCCCAGTAATTCTCAGTTGGAAATAGAAGGAAACAGTTCAG
GGCGACAGCTCAGTGAAGTCTTCATTCAGTTACCTTCAAGGAAAGAATTACCAGAATACTATGAATTAAT
TAGGAAGCCAGTGGATTTCAAAAAAATAAAGGAAAGGATTCGTAATCATAAGTACCGGAGCCTAGGCGAC
CTGGAGAAGGATGTCATGCTTCTCTGTCACAACGCTCAGACGTTCAACCTGGAGGGATCCCAGATCTATG
AAGACTCCATCGTCTTACAGTCAGTGTTTAAGAGTGCCCGGCAGAAAATTGCCAAAGAGGAAGAGAGTGA
GGATGAAAGCAATGAAGAGGAGGAAGAGGAAGATGAAGAAGAGTCAGAGTCCGAGGCAAAATCAGTCAAG
GTGAAAATTAAGCTCAATAAAAAAGATGACAAAGGCCGGGACAAAGGGAAAGGCAAGAAAAGGCCAAATC
GAGGAAAAGCCAAACCTGTAGTGAGCGATTTTGACAGCGATGAGGAGCAGGATGAACGTGAACAGTCAGA
AGGAAGTGGGACGGATGATGAGTGATCAGTATGGACCTTTTTCCTTGGTAGAACTGAATTCCTTCCTCCC
CTGTCTCATTTCTACCCAGTGAGTTCATTTGTCATATAGGCACTGGGTTGTTTCTATATCATCATCGTCT
ATAAACTAGCTTTAGGATAGTGCCAGACAAACATATGATATCATGGTGTAAAAAACACACACATACACAA
ATATTTGTAACATATTGTGACCAAATGGGCCTCAAAGATTCAGATTGAAACAAACAAAAAGCTTTTGATG
GAAAATATGTGGGTGGATAGTATATTTCTATGGGTGGGTCTAATTTGGTAACGGTTTGATTGTGCCTGGT
TTTATCACCTGTTCAGATGAGAAGATTTTTGTCTTTTGTAGCACTGATAACCAGGAGAAGCCATTAAAAG
CCACTGGTTATTTTATTTTTCATCAGGCAATTTTCGAGGTTTTTATTTGTTCGGTATTGTTTTTTTACAC
TGTGGTACATATAAGCAACTTTAATAGGTGATAAATGTACAGTAGTTAGATTTCACCTGCATATACATTT
TTCCATTTTATGCTCTATGATCTGAACAAAAGCTTTTTGAATTGTATAAGATTTATGTCTACTGTAAACA
TTGCTTAATTTTTTTGCTCTTGATTTAAAAAAAAGTTTTGTTGAAAGCGCTATTGAATATTGCAATCTAT
ATAGTGTATTGGATGGCTTCTTTTGTCACCCTGATCTCCTATGTTACCAATGTGTATCGTCTCCTTCTCC
CTAAAGTGTACTTAATCTTTGCTTTCTTTGCACAATGTCTTTGGTTGCAAGTCATAAGCCTGAGGCAAAT
AAAATTCCAGTAATTTCGAAGAATGTGGTGTTGGTGCTTTCCTAATAAAGAAATAATTTAGCTTGACAAA
AAAAAAAAAAAA.

The term “BRM” also refers to natural variants of the wild-type human BRM protein, such as proteins having at least 85% identity (e.g., 85%, 86%, 87%, 88%, 89%, 90%, 91%, 92%, 93%, 94%, 95%, 96%, 97%, 98%, 99%, 99.9% identity, or more) to an amino acid sequence of wild-type BRM, which is set forth in SEQ ID NO: 4 (Uniprot Accession No.: P51531; www.uniprot.org/uniprot/P51531.fasta).

SEQ ID NO: 4
MSTPTDPGAMPHPGPSPGPGPSPGPILGPSPGPGPSPGSVHSMMGPSPGP
PSVSHPMPTMGSTDFPQEGMHQMHKPIDGIHDKGIVEDIHCGSMKGTGMR
PPHPGMGPPQSPMDQHSQGYMSPHPSPLGAPEHVSSPMSGGGPTPPQMPP
SQPGALIPGDPQAMSQPNRGPSPFSPVQLHQLRAQILAYKMLARGQPLPE
TLQLAVQGKRTLPGLQQQQQQQQQQQQQQQQQQQQQQQPQQQPPQPQTQQ
QQQPALVNYNRPSGPGPELSGPSTPQKLPVPAPGGRPSPAPPAAAQPPAA
AVPGPSVPQPAPGQPSPVLQLQQKQSRISPIQKPQGLDPVEILQEREYRL
QARIAHRIQELENLPGSLPPDLRTKATVELKALRLLNFQRQLRQEVVACM
RRDTTLETALNSKAYKRSKRQTLREARMTEKLEKQQKIEQERKRRQKHQE
YLNSILQHAKDFKEYHRSVAGKIQKLSKAVATWHANTEREQKKETERIEK
ERMRRLMAEDEEGYRKLIDQKKDRRLAYLLQQTDEYVANLTNLVWEHKQA
QAAKEKKKRRRRKKKAEENAEGGESALGPDGEPIDESSQMSDLPVKVTHT
ETGKVLFGPEAPKASQLDAWLEMNPGYEVAPRSDSEESDSDYEEEDEEEE
SSRQETEEKILLDPNSEEVSEKDAKQIIETAKQDVDDEYSMQYSARGSQS
YYTVAHAISERVEKQSALLINGTLKHYQLQGLEWMVSLYNNNLNGILADE
MGLGKTIQTIALITYLMEHKRINGPYLIIVPLSTLSNWTYEFDKWAPSVV
KISYKGTPAMRRSLVPQLRSGKENVLLTTYEYIIKDKHILAKIRWKYMIV
DEGHRMKNHHCKLTQVLNTHYVAPRRILLTGTPLQNKLPELWALLNFLLP
TIFKSCSTFEQWFNAPFAMTGERVDLNEEETILIIRRLHKVLRPELLRRL
KKEVESQLPEKVEYVIKCDMSALQKILYRHMQAKGILLTDGSEKDKKGKG
GAKTLMNTIMQLRKICNHPYMFQHIEESFAEHLGYSNGVINGAELYRASG
KFELLDRILPKLRATNHRVLLFCQMTSLMTIMEDYFAFRNFLYLRLDGTT
KSEDRAALLKKENEPGSQYFIFLLSTRAGGLGLNLQAADTVVIFDSDWNP
HQDLQAQDRAHRIGQQNEVRVLRLCTVNSVEEKILAAAKYKLNVDQKVIQ
AGMFDQKSSSHERRAFLQAILEHEEENEEEDEVPDDETLNQMIARREEEF
DLFMRMDMDRRREDARNPKRKPRLMEEDELPSWIIKDDAEVERLTCEEEE
EKIFGRGSRQRRDVDYSDALTEKQWLRAIEDGNLEEMEEEVRLKKRKRRR
NVDKDPAKEDVEKAKKRRGRPPAEKLSPNPPKLTKQMNAIIDTVINYKDR
CNVEKVPSNSQLEIEGNSSGRQLSEVFIQLPSRKELPEYYELIRKPVDFK
KIKERIRNHKYRSLGDLEKDVMLLCHNAQTFNLEGSQIYEDSIVLQSVEK
SARQKIAKEEESEDESNEEEEEEDEEESESEAKSVKVKIKLNKKDDKGRD
KGKGKKRPNRGKAKPVVSDFDSDEEQDEREQSEGSGTDDE.

The term “cancer” refers to a condition caused by the proliferation of malignant neoplastic cells, such as tumors, neoplasms, carcinomas, sarcomas, leukemias, and lymphomas.

As used herein, a “combination therapy” or “administered in combination” means that two (or more) different agents or treatments are administered to a subject as part of a defined treatment regimen for a particular disease or condition. The treatment regimen defines the doses and periodicity of administration of each agent such that the effects of the separate agents on the subject overlap. In some embodiments, the delivery of the two or more agents is simultaneous or concurrent and the agents may be co-formulated. In some embodiments, the two or more agents are not co-formulated and are administered in a sequential manner as part of a prescribed regimen. In some embodiments, administration of two or more agents or treatments in combination is such that the reduction in a symptom, or other parameter related to the disorder is greater than what would be observed with one agent or treatment delivered alone or in the absence of the other. The effect of the two treatments can be partially additive, wholly additive, or greater than additive (e.g., synergistic). Sequential or substantially simultaneous administration of each therapeutic agent can be effected by any appropriate route including, but not limited to, oral routes, intravenous routes, intramuscular routes, and direct absorption through mucous membrane tissues. The therapeutic agents can be administered by the same route or by different routes. For example, a first therapeutic agent of the combination may be administered by intravenous injection while a second therapeutic agent of the combination may be administered orally.

The term “CTLA-4 inhibitor,” as used herein, refers to a compound such as an antibody capable of inhibiting the activity of the protein that in humans is encoded by the CTLA4 gene. Known CTLA-4 inhibitors include ipilimumab.

By “decreasing the activity of a BAF complex” is meant decreasing the level of an activity related to a BAF complex, or a related downstream effect. A non-limiting example of decreasing an activity of a BAF complex is Sox2 activation. The activity level of a BAF complex may be measured using any method known in the art, e.g., the methods described in Kadoch et al. Cell, 2013, 153, 71-85, the methods of which are herein incorporated by reference.

By “determining the level” of a protein or RNA is meant the detection of a protein or an RNA, by methods known in the art, either directly or indirectly. “Directly determining” means performing a process (e.g., performing an assay or test on a sample or “analyzing a sample” as that term is defined herein) to obtain the physical entity or value. “Indirectly determining” refers to receiving the physical entity or value from another party or source (e.g., a third-party laboratory that directly acquired the physical entity or value). Methods to measure protein level generally include, but are not limited to, western blotting, immunoblotting, enzyme-linked immunosorbent assay (ELISA), radioimmunoassay (RIA), immunoprecipitation, immunofluorescence, surface plasmon resonance, chemiluminescence, fluorescent polarization, phosphorescence, immunohistochemical analysis, matrix-assisted laser desorption/ionization time-of-flight (MALDI-TOF) mass spectrometry, liquid chromatography (LC)-mass spectrometry, microcytometry, microscopy, fluorescence activated cell sorting (FACS), and flow cytometry, as well as assays based on a property of a protein including, but not limited to, enzymatic activity or interaction with other protein partners. Methods to measure RNA levels are known in the art and include, but are not limited to, quantitative polymerase chain reaction (qPCR) and Northern blot analyses.

As used herein, the term “derivative” refers to naturally-occurring, synthetic, and semi-synthetic analogues of a compound, peptide, protein, or other substance described herein. A derivative of a compound, peptide, protein, or other substance described herein may retain or improve upon the biological activity of the original material.

By a “drug resistant” is meant a cancer that does not respond, or exhibits a decreased response to, one or more chemotherapeutic agents (e.g., any agent described herein). A cancer determined to be “resistant” to a drug, as used herein, refers to a cancer that is drug resistant, based on unresponsiveness or decreased responsiveness to a chemotherapeutic agent, or is predicted to be drug resistant based on a prognostic assay (e.g., a gene expression assay).

As used herein, the term “inhibiting BRM” and/or “inhibiting BRG1” refers to blocking or reducing the level or activity of the ATPase catalytic binding domain or the bromodomain of the protein. BRM and/or BRG1 inhibition may be determined using methods known in the art, e.g., a BRM and/or BRG1 ATPase assay, a Nano DSF assay, or a BRM and/or BRG1 Luciferase cell assay.

As used herein, the term “failed to respond to a prior therapy” or “refractory to a prior therapy,” refers to a cancer that progressed despite treatment with the therapy.

By “level” is meant a level of a protein, or mRNA encoding the protein, as compared to a reference. The reference can be any useful reference, as defined herein. By a “decreased level” or an “increased level” of a protein is meant a decrease or increase in protein level, as compared to a reference (e.g., a decrease or an increase by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 100%, about 150%, about 200%, about 300%, about 400%, about 500%, or more; a decrease or an increase of more than about 10%, about 15%, about 20%, about 50%, about 75%, about 100%, or about 200%, as compared to a reference; a decrease or an increase by less than about 0.01-fold, about 0.02-fold, about 0.1-fold, about 0.3-fold, about 0.5-fold, about 0.8-fold, or less; or an increase by more than about 1.2-fold, about 1.4-fold, about 1.5-fold, about 1.8-fold, about 2.0-fold, about 3.0-fold, about 3.5-fold, about 4.5-fold, about 5.0-fold, about 10-fold, about 15-fold, about 20-fold, about 30-fold, about 40-fold, about 50-fold, about 100-fold, about 1000-fold, or more). A level of a protein may be expressed in mass/vol (e.g., g/dL, mg/mL, μg/mL, ng/mL) or percentage relative to total protein or mRNA in a sample.

As used herein, the term “LXS196,” also known as IDE196, refers to the PKC inhibitor having the structure:

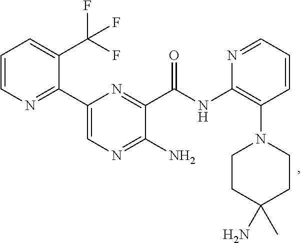 [SMILES: CC1(N)CCN(c2cccnc2NC(=O)c2nc(-c3ncccc3C(F)(F)F)cnc2N)CC1]

or a pharmaceutically acceptable salt thereof.

The term “MEK inhibitor,” as used herein, refers to a compound capable of inhibiting the activity of the mitogen-activated protein kinase enzyme MEK1 or MEK2. An MEK inhibitor may be, e.g., selumetinib, binimetinib, or tametinib.

As used herein, “metastatic nodule” refers to an aggregation of tumor cells in the body at a site other than the site of the original tumor.

As used herein, “metastatic cancer” refers to a tumor or cancer in which the cancer cells forming the tumor have a high potential to or have begun to, metastasize, or spread from one location to another location or locations within a subject, via the lymphatic system or via hematogenous spread, for example, creating secondary tumors within the subject. Such metastatic behavior may be indicative of malignant tumors. In some cases, metastatic behavior may be associated with an increase in cell migration and/or invasion behavior of the tumor cells.

Examples of cancers that can be defined as metastatic include, but are not limited to, lung cancer (e.g., non-small cell lung cancer), breast cancer, ovarian cancer, colorectal cancer, biliary tract cancer, bladder cancer, brain cancer including glioblastomas and medulloblastomas, cervical cancer, choriocarcinoma, endometrial cancer, esophageal cancer, gastric cancer, hematological neoplasms, multiple myeloma, leukemia, intraepithelial neoplasms, liver cancer, lymphomas, neuroblastomas, oral cancer, pancreatic cancer, prostate cancer, sarcoma, skin cancer including melanoma, basocellular cancer, squamous cell cancer, testicular cancer, stromal tumors, germ cell tumors, thyroid cancer, and renal cancer.

“Non-metastatic cell migration cancer” as used herein refers to cancers that do not migrate via the lymphatic system or via haematogenous spread.

“Percent (%) sequence identity” with respect to a reference polynucleotide or polypeptide sequence is defined as the percentage of nucleic acids or amino acids in a candidate sequence that are identical to the nucleic acids or amino acids in the reference polynucleotide or polypeptide sequence, after aligning the sequences and introducing gaps, if necessary, to achieve the maximum percent sequence identity. Alignment for purposes of determining percent nucleic acid or amino acid sequence identity can be achieved in various ways that are within the capabilities of one of skill in the art, for example, using publicly available computer software such as BLAST, BLAST-2, or Megalign software. Those skilled in the art can determine appropriate parameters for aligning sequences, including any algorithms needed to achieve maximal alignment over the full length of the sequences being compared. For example, percent sequence identity values may be generated using the sequence comparison computer program BLAST. As an illustration, the percent sequence identity of a given nucleic acid or amino acid sequence, A, to, with, or against a given nucleic acid or amino acid sequence, B, (which can alternatively be phrased as a given nucleic acid or amino acid sequence, A that has a certain percent sequence identity to, with, or against a given nucleic acid or amino acid sequence, B) is calculated as follows:

$$1000 multiplied by ( the fraction X / Y )$$

where X is the number of nucleotides or amino acids scored as identical matches by a sequence alignment program (e.g., BLAST) in that program's alignment of A and B, and where Y is the total number of nucleic acids in B. It will be appreciated that where the length of nucleic acid or amino acid sequence A is not equal to the length of nucleic acid or amino acid sequence B, the percent sequence identity of A to B will not equal the percent sequence identity of B to A.

The term “PD-1 inhibitor,” as used herein, refers to a compound such as an antibody capable of inhibiting the activity of the protein that in humans is encoded by the PDCD1 gene. Known PD-1 inhibitors include nivolumab, pembrolizumab, pidilizumab, BMS 936559, and atezolizumab.

The term “PD-L1 inhibitor,” as used herein, refers to a compound such as an antibody capable of inhibiting the activity of the protein that in humans is encoded by the CD274 gene. Known PD-L1 inhibitors include atezolizumab and durvalumab.

The term “pharmaceutical composition,” as used herein, represents a composition containing a compound described herein formulated with a pharmaceutically acceptable excipient and appropriate for administration to a mammal, for example a human. Typically, a pharmaceutical composition is manufactured or sold with the approval of a governmental regulatory agency as part of a therapeutic regimen for the treatment of disease in a mammal. Pharmaceutical compositions can be formulated, for example, for oral administration in unit dosage form (e.g., a tablet, capsule, caplet, gelcap, or syrup); for topical administration (e.g., as a cream, gel, lotion, or ointment); for intravenous administration (e.g., as a sterile solution free of particulate emboli and in a solvent system suitable for intravenous use); or in any other pharmaceutically acceptable formulation.

A “pharmaceutically acceptable excipient,” as used herein, refers to any ingredient other than the compounds described herein (for example, a vehicle capable of suspending or dissolving the active compound) and having the properties of being substantially nontoxic and non-inflammatory in a subject. Excipients may include, for example: antiadherents, antioxidants, binders, coatings, compression aids, disintegrants, dyes (colors), emollients, emulsifiers, fillers (diluents), film formers or coatings, flavors, fragrances, glidants (flow enhancers), lubricants, preservatives, printing inks, sorbents, suspending or dispersing agents, sweeteners, and waters of hydration.

As used herein, the term “pharmaceutically acceptable salt” means any pharmaceutically acceptable salt of a compound described herein. Pharmaceutically acceptable salts of any of the compounds described herein may include those that are within the scope of sound medical judgment, suitable for use in contact with the tissues of humans and animals without undue toxicity, irritation, allergic response and are commensurate with a reasonable benefit/risk ratio. Pharmaceutically acceptable salts are well known in the art. For example, pharmaceutically acceptable salts are described in: Berge et al., J. Pharmaceutical Sciences 66:1-19, 1977 and in Pharmaceutical Salts: Properties, Selection, and Use, (Eds. P. H. Stahl and C. G. Wermuth), Wiley-VCH, 2008. The salts can be prepared in situ during the final isolation and purification of the compounds described herein or separately, e.g., by reacting a free base group with a suitable organic acid.

The compounds of the invention may have ionizable groups so as to be capable of preparation as pharmaceutically acceptable salts. These salts may be, e.g., acid addition salts involving inorganic or organic acids or the salts may, in the case of acidic forms of the compounds of the invention be prepared from inorganic or organic bases. Frequently, the compounds are prepared or used as pharmaceutically acceptable salts prepared as addition products of pharmaceutically acceptable acids or bases. Suitable pharmaceutically acceptable acids and bases and methods for preparation of the appropriate salts are well-known in the art. Salts may be prepared from pharmaceutically acceptable non-toxic acids and bases including inorganic and organic acids and bases.

“Progression-free survival” as used herein, refers to the length of time during and after medication or treatment during which the disease being treated (e.g., cancer) does not get worse.

The term “PKC inhibitor,” as used herein, refers to a compound capable of inhibiting the activity of the protein kinase C. A PKC inhibitor may be, e.g., sotrastaurin or IDE196.

“Proliferation” as used in this application involves reproduction or multiplication of similar forms (cells) due to constituting (cellular) elements.

By “reducing the activity of BRG1 and/or BRM” is meant decreasing the level of an activity related to a BRG1 and/or BRM, or a related downstream effect. A non-limiting example of inhibition of an activity of BRG1 and/or BRM is decreasing the level of a BAF complex (e.g., GBAF) in a cell. The activity level of BRG1 and/or BRM may be measured using any method known in the art. In some embodiments, an agent which reduces the activity of BRG1 and/or BRM is a small molecule BRG1 and/or BRM inhibitor

By “reducing the level of BRG1 and/or BRM” is meant decreasing the level of BRG1 and/or BRM in a cell or subject. The level of BRG1 and/or BRM may be measured using any method known in the art.

By a “reference” is meant any useful reference used to compare protein or RNA levels. The reference can be any sample, standard, standard curve, or level that is used for comparison purposes. The reference can be a normal reference sample or a reference standard or level. A “reference sample” can be, for example, a control, e.g., a predetermined negative control value such as a “normal control” or a prior sample taken from the same subject; a sample from a normal healthy subject, such as a normal cell or normal tissue; a sample (e.g., a cell or tissue) from a subject not having a disease; a sample from a subject that is diagnosed with a disease, but not yet treated with a compound of the disclosure; a sample from a subject that has been treated by a compound of the disclosure; or a sample of a purified protein or RNA (e.g., any described herein) at a known normal concentration. By “reference standard or level” is meant a value or number derived from a reference sample. A “normal control value” is a predetermined value indicative of non-disease state, e.g., a value expected in a healthy control subject. Typically, a normal control value is expressed as a range (“between X and Y”), a high threshold (“no higher than X”), or a low threshold (“no lower than X”). A subject having a measured value within the normal control value for a particular biomarker is typically referred to as “within normal limits” for that biomarker. A normal reference standard or level can be a value or number derived from a normal subject not having a disease or disorder (e.g., cancer); a subject that has been treated with a compound of the disclosure. In preferred embodiments, the reference sample, standard, or level is matched to the sample subject sample by at least one of the following criteria: age, weight, sex, disease stage, and overall health. A standard curve of levels of a purified protein or RNA, e.g., any described herein, within the normal reference range can also be used as a reference.

As used herein, “slowing the spread of metastasis” refers to reducing or stopping the formation of new loci; or reducing, stopping, or reversing the tumor load.

As used herein, the term “subject” refers to any organism to which a composition in accordance with the disclosure may be administered, e.g., for experimental, diagnostic, prophylactic, and/or therapeutic purposes. Typical subjects include any animal (e.g., mammals such as mice, rats, rabbits, non-human primates, and humans). A subject may seek or be in need of treatment, require treatment, be receiving treatment, be receiving treatment in the future, or be a human or animal who is under care by a trained professional for a particular disease or condition.

As used herein, the terms “treat,” “treated,” or “treating” mean therapeutic treatment or any measures whose object is to slow down (lessen) an undesired physiological condition, disorder, or disease, or obtain beneficial or desired clinical results. Beneficial or desired clinical results include, but are not limited to, alleviation of symptoms; diminishment of the extent of a condition, disorder, or disease; stabilized (i.e., not worsening) state of condition, disorder, or disease; delay in onset or slowing of condition, disorder, or disease progression; amelioration of the condition, disorder, or disease state or remission (whether partial or total); an amelioration of at least one measurable physical parameter, not necessarily discernible by the subject; or enhancement or improvement of condition, disorder, or disease. Treatment includes eliciting a clinically significant response without excessive levels of side effects. Treatment also includes prolonging survival as compared to expected survival if not receiving treatment. In the context of treating cancer, treatment may include slowing the spread of metastasis and/or extending progression-free survival in a population of treated subjects as compared to a population of untreated subjects. Compounds of the disclosure may also be used to “prophylactically treat” or “prevent” a disorder, for example, in a subject at increased risk of developing the disorder.

Unless otherwise defined, all technical and scientific terms used herein have the same meaning as commonly understood by one of ordinary skill in the art to which this disclosure belongs. Methods and materials are described herein for use in the present disclosure; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

The details of one or more embodiments of the invention are set forth in the description below. Other features, objects, and advantages of the invention will be apparent from the description and from the claims.

### BRIEF DESCRIPTION OF THE DRAWINGS

FIG. 1A shows a Monte Carlo simulation plot of Compound A concentration (ng/mL) vs. time (days) for 10 mg QD dosing. IC₅₀ thresholds for both AML and UM are provided.

FIG. 1B shows a Monte Carlo simulation plot of Compound A concentration (ng/mL) vs. time (days) for 10 mg every other day dosing. IC₅₀ for both AML and UM are provided.

FIG. 1C shows a Monte Carlo simulation plot of Compound A concentration (ng/mL) vs. time (days) for 10 mg QD for 14 days, followed by one week without administration of Compound A. IC₅₀ for both AML and UM are provided.

FIG. 1D shows a Monte Carlo simulation plot of Compound A concentration (ng/mL) vs. time (days) for 10 mg QD for 7 days, followed by one week without administration of Compound A. IC₅₀ for both AML and UM are provided.

FIG. 2A shows a Monte Carlo simulation plot of log Compound A concentration (ng/mL) vs. time (days) for 10 mg QD dosing. IC₅₀ for both AML and UM are provided.

FIG. 2B shows a Monte Carlo simulation plot of log Compound A concentration (ng/mL) vs. time (days) for 10 mg every other day dosing. IC₅₀ for both AML and UM are provided.

FIG. 2C shows a Monte Carlo simulation plot of log Compound A concentration (ng/mL) vs. time (days) for 10 mg QD for 14 days, followed by one week without administration of Compound A. IC₅₀ for both AML and UM are provided.

FIG. 2D shows a Monte Carlo simulation plot of log Compound A concentration (ng/mL) vs. time (days) for 10 mg QD for 7 days, followed by one week without administration of Compound A. IC₅₀ for both AML and UM are provided.

### DETAILED DESCRIPTION

The present disclosure features compounds useful for the inhibition of BRG1 and/or BRM. These compounds may be used to modulate the activity of a BAF complex, for example, for the treatment of a BAF-related disorder, such as cancer. The compound may be, e.g., N-(1-((4-(6-(2,6-dimethylmorpholino)pyridin-2-yl)thiazol-2-yl)amino)-3-methoxy-1-oxopropan-2-yl)-1-(methylsulfonyl)-1H-pyrrole-3-carboxamide or a pharmaceutically acceptable salt thereof. This compound may have the following structure:

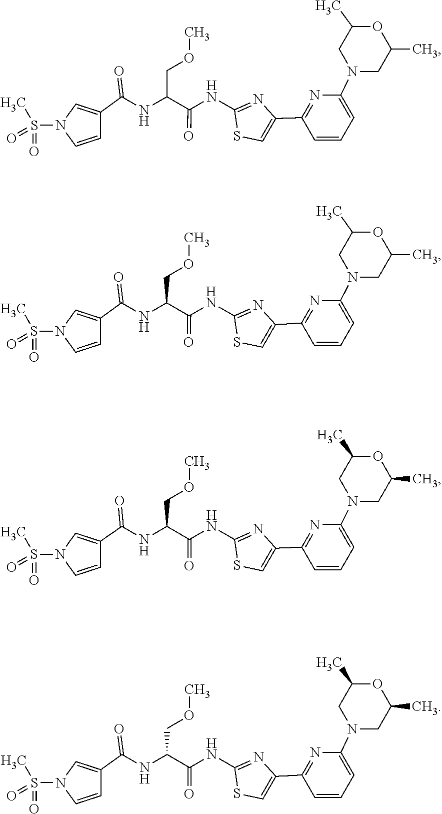 [SMILES: COCC(NC(=O)c1ccn(S(C)(=O)=O)c1)C(=O)Nc1nc(-c2cccc(N3CC(C)OC(C)C3)n2)cs1 ; COC[C@H](NC(=O)c1ccn(S(C)(=O)=O)c1)C(=O)Nc1nc(-c2cccc(N3CC(C)OC(C)C3)n2)cs1 ; COC[C@H](NC(=O)c1ccn(S(C)(=O)=O)c1)C(=O)Nc1nc(-c2cccc(N3C[C@@H](C)O[C@@H](C)C3)n2)cs1 ; COC[C@@H](NC(=O)c1ccn(S(C)(=O)=O)c1)C(=O)Nc1nc(-c2cccc(N3C[C@@H](C)O[C@@H](C)C3)n2)cs1]

or a pharmaceutically acceptable salt thereof.

Methods described herein may be of treating cancer in a subject in need thereof. Methods described herein may be of decreasing the level and/or activity of BRG1 and/or BRM in a subject. Methods described herein may be of treating a BAF complex-related disorder in a subject in need thereof. Methods described herein may be of treating a disorder related to a BRG1 loss of function mutation in a subject in need thereof. Methods described herein may be of decreasing the level and/or activity of a BAF complex in a cell of a subject. Methods described herein may be of inhibiting BRM in a cell of a subject. Methods described herein may be of inhibiting BRG1 in a cell of a subject. Methods described herein may be of inducing apoptosis in a cell in a subject.

The methods described herein may include the step of administering to the subject an effective amount of a compound, N-(1-((4-(6-(2,6-dimethylmorpholino)pyridin-2-yl)thiazol-2-yl)amino)-3-methoxy-1-oxopropan-2-yl)-1-(methylsulfonyl)-1H-pyrrole-3-carboxamide, or a pharmaceutically acceptable salt thereof, at least once daily for six to eight days, followed by six to eight days without administration of the compound, or a pharmaceutically acceptable salt thereof.

The methods described herein may include the step of administering to the subject an effective amount of a compound, N-(1-((4-(6-(2,6-dimethylmorpholino)pyridin-2-yl)thiazol-2-yl)amino)-3-methoxy-1-oxopropan-2-yl)-1-(methylsulfonyl)-1H-pyrrole-3-carboxamide, or a pharmaceutically acceptable salt thereof, at least once daily for thirteen to fifteen days, followed by six to eight days without administration of the compound, or a pharmaceutically acceptable salt thereof.

The methods described herein may include the step of administering to the subject an effective amount of a compound, N-(1-((4-(6-(2,6-dimethylmorpholino)pyridin-2-yl)thiazol-2-yl)amino)-3-methoxy-1-oxopropan-2-yl)-1-(methylsulfonyl)-1H-pyrrole-3-carboxamide, or a pharmaceutically acceptable salt thereof, at least once daily for six to fifteen days, followed by six to eight days without administration of the compound, or a pharmaceutically acceptable salt thereof.

The methods described herein may include the step of administering to the subject an effective amount of a compound, N-(1-((4-(6-(2,6-dimethylmorpholino)pyridin-2-yl)thiazol-2-yl)amino)-3-methoxy-1-oxopropan-2-yl)-1-(methylsulfonyl)-1H-pyrrole-3-carboxamide, or a pharmaceutically acceptable salt thereof, at least once daily for seven days, followed by seven days without administration of the compound, or a pharmaceutically acceptable salt thereof.

The methods described herein may include the step of administering to the subject an effective amount of a compound, N-(1-((4-(6-(2,6-dimethylmorpholino)pyridin-2-yl)thiazol-2-yl)amino)-3-methoxy-1-oxopropan-2-yl)-1-(methylsulfonyl)-1H-pyrrole-3-carboxamide, or a pharmaceutically acceptable salt thereof, at least once daily for fourteen days, followed by seven days without administration of the compound, or a pharmaceutically acceptable salt thereof.

The methods described herein may include the step of administering to the subject an effective amount of a compound, N-(1-((4-(6-(2,6-dimethylmorpholino)pyridin-2-yl)thiazol-2-yl)amino)-3-methoxy-1-oxopropan-2-yl)-1-(methylsulfonyl)-1H-pyrrole-3-carboxamide, or a pharmaceutically acceptable salt thereof, at least once daily for seven to fourteen days, followed by seven days without administration of the compound, or a pharmaceutically acceptable salt thereof.

Methods for the synthesizing the compounds are described in International Application No. PCT/US2021/015876, the content of which is incorporated herein by reference in its entirety.

### Method of Treatment

The compounds described herein are useful in the methods of the disclosure and, while not bound by theory, are believed to exert their ability to modulate the level, status, and/or activity of a BAF complex, i.e., by inhibiting the activity of the BRG1 and/or BRM proteins within the BAF complex in a mammal. BAF complex-related disorders include, but are not limited to, BRG1 loss of function mutation-related disorders.

An aspect of the present disclosure relates to methods of treating disorders related to BRG1 loss of function mutations such as cancer (e.g., non-small cell lung cancer, colorectal cancer, bladder cancer, cancer of unknown primary, glioma, breast cancer, melanoma, non-melanoma skin cancer, endometrial cancer, or penile cancer) in a subject in need thereof. In some embodiments, the present invention relates to methods of treating melanoma (e.g., uveal melanoma), prostate cancer, breast cancer, bone cancer, renal cell carcinoma, or a hematologic cancer.

In some embodiments, the methods of the present disclosure result in one or more (e.g., two or more, three or more, four or more) of: (a) reduced tumor size, (b) reduced rate of tumor growth, (c) increased tumor cell death (d) reduced tumor progression, (e) reduced number of metastases, (f) reduced rate of metastasis, (g) decreased tumor recurrence (h) increased survival of subject, (i) increased progression free survival of subject.

Treating cancer can result in a reduction in size or volume of a tumor. For example, after treatment, tumor size is reduced by 5% or greater (e.g., 10%, 20%, 30%, 40%, 50%, 60%, 70%, 80%, 90%, or greater) relative to its size prior to treatment. Size of a tumor may be measured by any reproducible means of measurement. For example, the size of a tumor may be measured as a diameter of the tumor.

Treating cancer may further result in a decrease in number of tumors. For example, after treatment, tumor number is reduced by 5% or greater (e.g., 10%, 20%, 30%, 40%, 50%, 60%, 70%, 80%, 90%, or greater) relative to number prior to treatment. Number of tumors may be measured by any reproducible means of measurement, e.g., the number of tumors may be measured by counting tumors visible to the naked eye or at a specified magnification (e.g., 2×, 3×, 4×, 5×, 10×, or 50×).

Treating cancer can result in a decrease in number of metastatic nodules in other tissues or organs distant from the primary tumor site. For example, after treatment, the number of metastatic nodules is reduced by 5% or greater (e.g., 10%, 20%, 30%, 40%, 50%, 60%, 70%, 80%, 90% or greater) relative to number prior to treatment. The number of metastatic nodules may be measured by any reproducible means of measurement. For example, the number of metastatic nodules may be measured by counting metastatic nodules visible to the naked eye or at a specified magnification (e.g., 2×, 10×, or 50×).

Treating cancer can result in an increase in average survival time of a population of subjects treated according to the present invention in comparison to a population of untreated subjects. For example, the average survival time is increased by more than 30 days (more than 60 days, 90 days, or 120 days). An increase in average survival time of a population may be measured by any reproducible means. An increase in average survival time of a population may be measured, for example, by calculating for a population the average length of survival following initiation of treatment with the compound of the invention. An increase in average survival time of a population may also be measured, for example, by calculating for a population the average length of survival following completion of a first round of treatment with a pharmaceutically acceptable salt of the invention.

Treating cancer can also result in a decrease in the mortality rate of a population of treated subjects in comparison to an untreated population. For example, the mortality rate is decreased by more than 2% (e.g., more than 5%, 10%, or 25%). A decrease in the mortality rate of a population of treated subjects may be measured by any reproducible means, for example, by calculating for a population the average number of disease-related deaths per unit time following initiation of treatment with a pharmaceutically acceptable salt of the invention. A decrease in the mortality rate of a population may also be measured, for example, by calculating for a population the average number of disease-related deaths per unit time following completion of a first round of treatment with a pharmaceutically acceptable salt of the invention.

Exemplary cancers that may be treated by the invention include, but are not limited to, non-small cell lung cancer, small-cell lung cancer, colorectal cancer, bladder cancer, glioma, breast cancer, melanoma, non-melanoma skin cancer, endometrial cancer, esophagogastric cancer, esophageal cancer, pancreatic cancer, hepatobiliary cancer, soft tissue sarcoma, ovarian cancer, head and neck cancer, renal cell carcinoma, bone cancer, non-Hodgkin lymphoma, prostate cancer, embryonal tumor, germ cell tumor, cervical cancer, thyroid cancer, salivary gland cancer, gastrointestinal neuroendocrine tumor, uterine sarcoma, gastrointestinal stromal tumor, CNS cancer, thymic tumor, Adrenocortical carcinoma, appendiceal cancer, small bowel cancer, hematologic cancer, and penile cancer.

In some embodiments, the cancer expresses BRG1 and/or BRM protein and/or the cell or subject has been identified as expressing BRG1 and/or BRM. In some embodiments, the cancer expresses BRG1 protein and/or the cell or subject has been identified as expressing BRG1. In some embodiments, the cancer expresses BRM protein and/or the cell or subject has been identified as expressing BRM. In some embodiments, the subject or cancer has and/or has been identified as having a BRG1 loss of function mutation. In some embodiments, the subject or cancer has and/or has been identified as having a BRM loss of function mutation.

In some embodiments, the cancer has or has been determined to have one or more BRG1 mutations (e.g., homozygous mutations). In some embodiments, the one or more BRG1 mutations includes a mutation in the ATPase catalytic domain of the protein. In some embodiments, the one or more BRG1 mutations include a deletion at the C-terminus of BRG1.

In some embodiments, the cancer does not have, or has been determined not to have, an epidermal growth factor receptor (EGFR) mutation. In some embodiments of any of the foregoing methods, the cancer does not have, or has been determined not to have, an anaplastic lymphoma kinase (ALK) driver mutation. In some embodiments of any of the foregoing methods, the cancer has, or has been determined to have, a KRAS mutation.

In some embodiments, the cancer has, or has been determined to have, a mutation in GNAQ. In some embodiments, the metastatic uveal melanoma has, or has been determined to have, a mutation in GNA11. In some embodiments, the metastatic uveal melanoma has, or has been determined to have, a mutation in PLCB4. In some embodiments, the cancer has, or has been determined to have, a mutation in CYSLTR2. In some embodiments, the cancer has, or has been determined to have, a mutation in BAP1. In some embodiments, the metastatic uveal melanoma has, or has been determined to have, a mutation in SF361. In some embodiments, the cancer has, or has been determined to have, a mutation in EIF1AX. In some embodiments, the metastatic uveal melanoma has, or has been determined to have, a TFE3 translocation. In some embodiments, the cancer has, or has been determined to have, a TFEB translocation. In some embodiments, the metastatic uveal melanoma has, or has been determined to have, a MITF translocation. In some embodiments, the cancer has, or has been determined to have, an EZH2 mutation. In some embodiments, the cancer has, or has been determined to have, a SUZ12 mutation. In some embodiments, the cancer has, or has been determined to have, an EED mutation.

The dosage of the compounds of the disclosure, and/or compositions comprising a compound of the disclosure, can vary depending on many factors, such as the pharmacodynamic properties of the compound; the mode of administration; the age, health, and weight of the recipient; the nature and extent of the symptoms; the frequency of the treatment, and the type of concurrent treatment, if any; and the clearance rate of the compound in the animal to be treated. One of skill in the art can determine the appropriate dosage based on the above factors. The compounds of the disclosure may be administered initially in a suitable dosage that may be adjusted as required, depending on the clinical response.

In some embodiments, a compound or pharmaceutically acceptable salt of the disclosure is administered in one or more cycles. Each cycle typically includes a period of six to fifteen days during which an effective amount of a compound or pharmaceutically acceptable salt of the disclosure is administered to the subject at least once daily, followed by a period of six to eight days during which the compound or pharmaceutically acceptable salt thereof is not administered.

In some embodiments, each cycle includes a period of six to eight days during which an effective amount of a compound or pharmaceutically acceptable salt of the disclosure is administered to the subject at least once daily, followed by a period of six to eight days during which the compound or pharmaceutically acceptable salt thereof is not administered.

In some embodiments, each cycle includes a period of thirteen to fifteen days during which an effective amount of a compound or pharmaceutically acceptable salt of the disclosure is administered to the subject at least once daily, followed by a period of six to eight days during which the compound or pharmaceutically acceptable salt thereof is not administered.

In some embodiments, each cycle includes a period of seven days during which an effective amount of the compound or pharmaceutically acceptable salt thereof is administered to the subject at least once daily, followed by a period of seven days during which the compound or pharmaceutically acceptable salt thereof is not administered. In some embodiments, the compound or pharmaceutically acceptable salt thereof is administered in two or more cycles. In some embodiments, the compound or pharmaceutically acceptable salt thereof is administered in three or more cycles. In some embodiments, the compound or pharmaceutically acceptable salt thereof is administered in four or more cycles.

In some embodiments, each cycle includes a period of fourteen days during which an effective amount of the compound or pharmaceutically acceptable salt thereof is administered to the subject at least once daily, followed by a period of seven days during which the compound or pharmaceutically acceptable salt thereof is not administered.

In some embodiments, each cycle includes a period of seven to fourteen days during which an effective amount of the compound or pharmaceutically acceptable salt thereof is administered to the subject at least once daily, followed by a period of seven days during which the compound or pharmaceutically acceptable salt thereof is not administered.

In some embodiments, a compound or pharmaceutically acceptable salt of the disclosure is administered in total dose of between about 5.0 mg and about 20 mg per day. In some embodiments, a compound or pharmaceutically acceptable salt of the disclosure is administered in total dose of between about 5.0 mg and about 15 mg per day. In some embodiments, a compound or pharmaceutically acceptable salt of the disclosure is administered in total dose of between about 5.0 mg and about 10 mg per day. In some embodiments, a compound or pharmaceutically acceptable salt of the disclosure is administered in total dose of between about 5.0 mg and about 7.5 mg per day. In some embodiments, a compound or pharmaceutically acceptable salt of the disclosure is administered in total dose of between about 7.5 mg and about 20 mg per day. In some embodiments, a compound or pharmaceutically acceptable salt of the disclosure is administered in total dose of between about 7.5 mg and about 15 mg per day. In some embodiments, a compound or pharmaceutically acceptable salt of the disclosure is administered in total dose of between about 7.5 mg and about 10 mg per day. In some embodiments, a compound or pharmaceutically acceptable salt of the disclosure is administered in total dose of between about 10 mg and about 20 mg per day. In some embodiments, a compound or pharmaceutically acceptable salt of the disclosure is administered in total dose of between about 10 mg and about 15 mg per day. In some embodiments, a compound or pharmaceutically acceptable salt of the disclosure is administered in total dose of between about 15 mg and about 20 mg per day. In some embodiments, a compound or pharmaceutically acceptable salt of the disclosure is administered in total dose of about 5.0 mg per day. In some embodiments, a compound or pharmaceutically acceptable salt of the disclosure is administered in total dose of about 7.5 mg per day. In some embodiments, a compound or pharmaceutically acceptable salt of the disclosure is administered in total dose of about 10 mg per day. In some embodiments, a compound or pharmaceutically acceptable salt of the disclosure is administered in total dose of about 15 mg per day. In some embodiments, a compound or pharmaceutically acceptable salt of the disclosure is administered in total dose of about 20 mg per day.

In some embodiments, a compound or pharmaceutically acceptable salt of the disclosure is administered once daily.

Administration of a compound or pharmaceutically acceptable salt of the disclosure may be interrupted (i.e., a dose hold) if the subject exhibits dose limited toxicity.

Dose limiting toxicity is characterized by conditions including, but are not limited to, hyperbilirubinemia (e.g., Grade 3, Grade 4, or Grade 5 hyperbilirubinemia) and keratitis (e.g., Grade 1, Grade 2, Grade 3, Grade 4, or Grade 5 keratitis). Administration of the compound or pharmaceutically acceptable salt may be interrupted for a duration sufficient for the dose limiting to resolved or improved, e.g., Grade 3 hyperbilirubinemia to Grade 1 or Grade 2 hyperbilirubinemia, or Grade 3 keratitis to Grade 1 or Grade 2 keratitis. If administration of the compound or pharmaceutically acceptable salt of the disclosure is interrupted, its administration may be resumed.

In some embodiments of any of methods disclosed herein, the cancer is drug resistant (e.g., the cancer has been determined to be resistant, or likely to be resistant, to chemotherapeutic or cytotoxic agents such as by genetic markers, or is likely to be resistant, to chemotherapeutic or cytotoxic agents such as a cancer that has failed to respond to a chemotherapeutic or cytotoxic agent) and/or has failed to respond to a prior therapy (e.g., a chemotherapeutic or cytotoxic agent, immunotherapy, surgery, radiotherapy, thermotherapy, or photocoagulation, or a combination thereof).

In some embodiments, the cancer is resistant to and/or has failed to respond to vemurafenib, dacarbazine, a CTLA4 inhibitor, a PD1 inhibitor, interferon therapy, a BRAF inhibitor, a MEK inhibitor, radiotherapy, temozolomide, irinotecan, a CAR-T therapy, Herceptin® (trastuzumab), Perjeta® (pertuzumab), tamoxifen, Xeloda® (capecitabine), platinum agents such as carboplatin, taxanes such as paclitaxel and docetaxel, ALK inhibitors, MET inhibitors, Alimta (pemetrexed), Abraxane, doxorubicin, gemcitabine, Avastin®, Halaven®, neratinib, a PARP inhibitor, brilanestrant, an mTOR inhibitor, topotecan, Gemzar (gemcitabine HCl), a VEGFR2 inhibitor, a folate receptor antagonist, demcizumab, fosbretabulin, or a PDL1 inhibitor, or combinations thereof.

In some embodiments of any of the foregoing methods, the cancer is resistant to and/or has failed to respond to dacarbazine, temozolomide, cisplatin, treosulfan, fotemustine, IMCgp100, a CTLA-4 inhibitor (e.g., ipilimumab), a PD-1 inhibitor (e.g., nivolumab or pembrolizumab), a PD-L1 inhibitor (e.g., atezolizumab, avelumab, or durvalumab), a mitogen-activated protein kinase (MEK) inhibitor (e.g., selumetinib, binimetinib, or tametinib), and/or a protein kinase C (PKC) inhibitor (e.g., sotrastaurin or IDE196).

In some embodiments of any of the foregoing methods, the cancer is resistant to and/or failed to respond to a previously administered therapeutic used for the treatment of uveal melanoma, e.g., a MEK inhibitor or PKC inhibitor. For example, in some embodiments, the cancer is resistant to and/or failed to respond to a mitogen-activated protein kinase (MEK) inhibitor (e.g., selumetinib, binimetinib, or tametinib), and/or a protein kinase C (PKC) inhibitor (e.g., sotrastaurin or IDE196).

In some embodiments of any of the foregoing methods, the method or effective amount reduces the level and/or activity of BRG1 by at least 5% (e.g., at least 6%, at least 7%, at least 8%, at least 9%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 99%) as compared to a reference.

In some embodiments of any of the foregoing methods, the method or effective amount reduces the level and/or activity of BRG1 by at least 5% (e.g., at least 6%, at least 7%, at least 8%, at least 9%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 99%) as compared to a reference for at least 12 hours (e.g., at least 14 hours, at least 16 hours, at least 18 hours, at least 20 hours, at least 22 hours, at least 24 hours, at least 30 hours, at least 36 hours, at least 48 hours, at least 72 hours, at least 4 days, at least 5 days, at least 6 days, at least 7 days, at least 14 days, at least 21 days, at least 28 days, or more).

In some embodiments of any of the foregoing methods, the method or effective amount reduces the level and/or activity of BRM by at least 5% (e.g., at least 6%, at least 7%, at least 8%, at least 9%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 99%) as compared to a reference.

In some embodiments of any of the foregoing methods, the method or effective amount reduces the level and/or activity of BRM by at least 5% (e.g., at least 6%, at least 7%, at least 8%, at least 9%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 99%) as compared to a reference for at least 12 hours (e.g., at least 14 hours, at least 16 hours, at least 18 hours, at least 20 hours, at least 22 hours, at least 24 hours, at least 30 hours, at least 36 hours, at least 48 hours, at least 72 hours, at least 4 days, at least 5 days, at least 6 days, at least 7 days, at least 14 days, at least 21 days, at least 28 days, or more).

### Combination Formulations and Uses Thereof

The compounds of the disclosure can be combined with one or more therapeutic agents. In particular, the therapeutic agent can be one that treats or prophylactically treats metastatic uveal melanoma.

### Combination Therapies

A compound of the disclosure can be used alone or in combination with an additional therapeutic agent, e.g., other agents that treat cancer or symptoms associated therewith, or in combination with other types of treatment to treat cancer. In combination treatments, the dosages of one or more of the therapeutic compounds may be reduced from standard dosages when administered alone. For example, doses may be determined empirically from drug combinations and permutations or may be deduced by isobolographic analysis (e.g., Black et al., Neurology 65: S3-S6, 2005). In this case, dosages of the compounds, when combined, should provide a therapeutic effect.

In some embodiments, the second therapeutic agent is a chemotherapeutic agent (e.g., a cytotoxic agent or other chemical compound useful in the treatment of cancer). These include alkylating agents, antimetabolites, folic acid analogs, pyrimidine analogs, purine analogs and related inhibitors, vinca alkaloids, epipodophyllotoxins, antibiotics, L-Asparaginase, topoisomerase inhibitors, interferons, platinum coordination complexes, anthracenedione substituted urea, methyl hydrazine derivatives, adrenocortical suppressant, adrenocorticosteroids, progestins, estrogens, antiestrogen, androgens, antiandrogen, and gonadotropin-releasing hormone analog. Also included is 5-fluorouracil (5-FU), leucovorin (LV), irinotecan, oxaliplatin, capecitabine, paclitaxel and docetaxel. Non-limiting examples of chemotherapeutic agents include alkylating agents such as thiotepa and cyclophosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, triethylenephosphoramide, triethylenethiophosphoramide, and trimethylolmelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; sarcodictyin; spongistatin; nitrogen mustards such as chlorambucil, chlornaphazine, cyclophosphamide, estramustine, ifosfamide, mechlorethamine, mechlorethamine oxide hydrochloride, melphalan, novembichin, phenesterine, prednimustine, trofosfamide, and uracil mustard; nitrosoureas such as carmustine, chlorozotocin, fotemustine, lomustine, nimustine, and ranimustine; antibiotics such as the enediyne antibiotics (e.g., calicheamicin, especially calicheamicin gamma and calicheamicin omega (see, e.g., Agnew, Chem. Intl. Ed Engl. 33:183-186 (1994)); dynemicin, including dynemicin A; bisphosphonates, such as clodronate; an esperamicin (as well as neocarzinostatin chromophore and related chromoprotein enediyne antibiotic chromophores), aclacinomycin, actinomycin, authramycin, azaserine, bleomycin, cactinomycin, carubicin, carzinophilin, chromomycin, dactinomycin, daunorubicin, detorubicin, 6-diazo-5-oxo-L-norleucine, Adriamycin® (doxorubicin, including morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, porfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as folinic acid; aceglatone; aldophosphamide glycoside; aminolevulinic acid; eniluracil; amsacrine; bestrabucil; bisantrene; edatraxate; defofamine; demecolcine; diaziquone; eflornithine; elliptinium acetate; an epothilone; etoglucid; gallium nitrate; hydroxyurea; lentinan; lonidainine; maytansinoids such as maytansine and anatoxins; mitoguazone; mitoxantrone; mopidamol; nitraerine; pentostatin; phenamet; pirarubicin; losoxantrone; podophyllinic acid; 2-ethylhydrazide; procarbazine; PSK® polysaccharide complex (JHS Natural Products, Eugene, Oreg.); razoxane; rhizoxin; sizofuran; spirogermanium; tenuazonic acid; triaziquone; 2,2′,2″-trichlorotriethylamine; trichothecenes (especially T-2 toxin, verrucarin A, roridin A, and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g., Taxol® paclitaxel (Bristol-Myers Squibb Oncology, Princeton, N.J.), ABraxane®, cremophor-free, albumin-engineered nanoparticle formulation of paclitaxel (American Pharmaceutical Partners, Schaumberg, Ill.), and Taxotere® docetaxel (Rhone-Poulenc Rorer, Antony, France); chlorambucil; Gemzar® gemcitabine; 6-thioguanine; mercaptopurine; methotrexate; platinum coordination complexes such as cisplatin, oxaliplatin and carboplatin; vinblastine; platinum; etoposide (VP-16); ifosfamide; mitoxantrone; vincristine; Navelbine® vinorelbine; novantrone; teniposide; edatrexate; daunomycin; aminopterin; Xeloda®; ibandronate; irinotecan (e.g., CPT-11); topoisomerase inhibitor RFS 2000; difluoromethylornithine (DMFO); retinoids such as retinoic acid; capecitabine; and pharmaceutically acceptable salts, acids or derivatives of any of the above. Two or more chemotherapeutic agents can be used in a cocktail to be administered in combination with the first therapeutic agent described herein. Suitable dosing regimens of combination chemotherapies are known in the art and described in, for example, Saltz et al. (1999) Proc ASCO 18:233a and Douillard et al. (2000) Lancet 355:1041-7.

In some embodiments, the second therapeutic agent is a therapeutic agent which is a biologic such a cytokine (e.g., interferon or an interleukin (e.g., IL-2)) used in cancer treatment. In some embodiments the biologic is an anti-angiogenic agent, such as an anti-VEGF agent, e.g., bevacizumab (Avastin®). In some embodiments the biologic is an immunoglobulin-based biologic, e.g., a monoclonal antibody (e.g., a humanized antibody, a fully human antibody, an Fc fusion protein or a functional fragment thereof) that agonizes a target to stimulate an anti-cancer response or antagonizes an antigen important for cancer. Such agents include Rituxan® (Rituximab); Zenapax (Daclizumab); Simulect (Basiliximab); Synagis (Palivizumab); Remicade® (Infliximab); Herceptin (Trastuzumab); Mylotarg (Gemtuzumab ozogamicin); Campath (Alemtuzumab); Zevalin® (Ibritumomab tiuxetan); Humira® (Adalimumab); Xolair® (Omalizumab); Bexxar (Tositumomab-1-131); Raptiva® (Efalizumab); Erbitux (Cetuximab); Avastin® (Bevacizumab); Tysabri® (Natalizumab); Actemra® (Tocilizumab); Vectibix® (Panitumumab); Lucentis® (Ranibizumab); Soliris® (Eculizumab); Cimzia® (Certolizumab pegol); Simponi® (Golimumab); Ilaris® (Canakinumab); Stelara® (Ustekinumab); Arzerra® (Ofatumumab); Prolia® (Denosumab); Numax (Motavizumab); ABThrax (Raxibacumab); Benlysta (Belimumab); Yervoy® (Ipilimumab); Adcetris® (Brentuximab Vedotin); Perjeta® (Pertuzumab); Kadcyla® (Ado-trastuzumab emtansine); and Gazyva® (Obinutuzumab). Also included are antibody-drug conjugates.

The second agent may be a therapeutic agent which is a non-drug treatment. For example, the second therapeutic agent is radiation therapy, cryotherapy, hyperthermia and/or surgical excision of tumor tissue.

The second agent may be a checkpoint inhibitor. In one embodiment, the inhibitor of checkpoint is an inhibitory antibody (e.g., a monospecific antibody such as a monoclonal antibody). The antibody may be, e.g., humanized or fully human. In some embodiments, the inhibitor of checkpoint is a fusion protein, e.g., an Fc-receptor fusion protein. In some embodiments, the inhibitor of checkpoint is an agent, such as an antibody, which interacts with a checkpoint protein. In some embodiments, the inhibitor of checkpoint is an agent, such as an antibody, which interacts with the ligand of a checkpoint protein. In some embodiments, the inhibitor of checkpoint is an inhibitor (e.g., an inhibitory antibody or small molecule inhibitor) of CTLA-4 (e.g., an anti-CTLA4 antibody such as ipilimumab/Yervoy or tremelimumab). In some embodiments, the inhibitor of checkpoint is an inhibitor (e.g., an inhibitory antibody or small molecule inhibitor) of PD-1 (e.g., nivolumab/Opdivo®; pembrolizumab/Keytruda®; pidilizumab/CT-011). In some embodiments, the inhibitor of checkpoint is an inhibitor (e.g., an inhibitory antibody or small molecule inhibitor) of PDL1 (e.g., MPDL3280A/RG7446; MED14736; MSB0010718C; BMS 936559). In some embodiments, the inhibitor of checkpoint is an inhibitor (e.g., an inhibitory antibody or Fc fusion or small molecule inhibitor) of PDL2 (e.g., a PDL2/Ig fusion protein such as AMP 224). In some embodiments, the inhibitor of checkpoint is an inhibitor (e.g., an inhibitory antibody or small molecule inhibitor) of B7-H3 (e.g., MGA271), B7-H4, BTLA, HVEM, TIM3, GAL9, LAG3, VISTA, KIR, 2B4, CD160, CGEN-15049, CHK1, CHK2, A2aR, B-7 family ligands, or a combination thereof.

In some embodiments, the compound of the disclosure is used in combination with another anti-cancer therapy used for the treatment of uveal melanoma such as surgery, a MEK inhibitor, and/or a PKC inhibitor, or a combination thereof. For example, in some embodiments, the method further comprises performing surgery prior to, subsequent to, or at the same time as administration of the compound of the disclosure. In some embodiments, the method further comprises administration of a MEK inhibitor (e.g., selumetinib, binimetinib, or tametinib) and/or a PKC inhibitor (e.g., sotrastaurin or IDE196) prior to, subsequent to, or at the same time as administration of the compound of the disclosure.

In any of the combination embodiments described herein, the first and second therapeutic agents are administered simultaneously or sequentially, in either order. The first therapeutic agent may be administered immediately, up to 1 hour, up to 2 hours, up to 3 hours, up to 4 hours, up to 5 hours, up to 6 hours, up to 7 hours, up to, 8 hours, up to 9 hours, up to 10 hours, up to 11 hours, up to 12 hours, up to 13 hours, 14 hours, up to hours 16, up to 17 hours, up 18 hours, up to 19 hours up to 20 hours, up to 21 hours, up to 22 hours, up to 23 hours up to 24 hours or up to 1-7, 1-14, 1-21 or 1-30 days before or after the second therapeutic agent.

In some embodiments, the compound of the disclosure is used in combination with another anti-cancer therapy used for the treatment of uveal melanoma such as surgery, a MEK inhibitor, and/or a PKC inhibitor, or combinations thereof. For example, in some embodiments, the method further comprises performing surgery prior to, subsequent to, or at the same time as administration of the compound of the invention. In some embodiments, the method further comprises administration of a MEK inhibitor (e.g., selumetinib, binimetinib, or tametinib) and/or a PKC inhibitor (e.g., sotrastaurin or IDE196) prior to, subsequent to, or at the same time as administration of the compound of the disclosure.

In some embodiments, the anticancer therapy and the compound of the disclosure are administered within 28 days (e.g., within 21 days, within 14 days, or within 7 days) of each other and each in an amount that, when combined, is effective to treat the subject.

### Pharmaceutical Compositions

The compounds of the present disclosure may be formulated into pharmaceutical compositions for administration to human subjects in a biologically compatible form suitable for administration in vivo. Pharmaceutical compositions typically include an active agent as described herein and a physiologically acceptable excipient (e.g., a pharmaceutically acceptable excipient). Formulation principles for the compounds disclosed herein may be those described, e.g., in WO 2020/160180, the disclosure of which is incorporated by reference herein in its entirety.

The compounds of the disclosure may be administered, for example, by oral, parenteral, buccal, sublingual, nasal, rectal, patch, pump, or transdermal administration and the pharmaceutical compositions formulated accordingly. Parenteral administration includes intravenous, intraperitoneal, subcutaneous, intramuscular, transepithelial, nasal, intrapulmonary, intrathecal, rectal, and topical modes of administration. Parenteral administration may be by continuous infusion over a selected period of time. Preferably, the compound is administered orally.

Suitable pharmaceutical carriers, as well as pharmaceutical necessities for use in pharmaceutical formulations, are described in Remington: The Science and Practice of Pharmacy, 21^{st} Ed., Gennaro, Ed., Lippencott Williams & Wilkins (2005), a well-known reference text in this field, and in the USP/NF (United States Pharmacopeia and the National Formulary).

The compound described herein may be formulated into a unit dosage form for oral administration (e.g., a capsule) as described in Table 1. The API in Table 1 is a compound of the following structure:

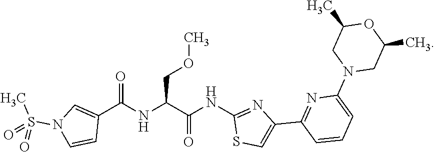 [SMILES: COC[C@H](NC(=O)c1ccn(S(C)(=O)=O)c1)C(=O)Nc1nc(-c2cccc(N3C[C@@H](C)O[C@@H](C)C3)n2)cs1]

TABLE 1

| | | 2.5 mg Capsule | | 20 mg Capsule | |
|---|---|---|---|---|---|
| | | Amount | | Amount | |
| | | per | | per | |
| | | Capsule | Content | Capsule | Content |
| Component | Function | (mg) | (%) | (mg) | (%) |
| API | Active | 2.50 | 2.63 | 20.00 | 17.39 |
| | ingredient | | | | |
| Microcrystalline | Filler | 48.24 | 50.78 | 48.20 | 41.91 |
| cellulose | | | | | |
| Mannitol | Filler | 36.18 | 38.08 | 36.95 | 32.13 |
| Croscarmellose | Disintegrant | 4.75 | 5.00 | 5.80 | 5.04 |
| Sodium | | | | | |
| Sodium lauryl | Wetting | 0.95 | 1.00 | 1.15 | 1.00 |
| sulfate | agent | | | | |
| Colloidal silicon | Glidant | 1.90 | 2.00 | 2.30 | 2.00 |
| dioxide | | | | | |
| Magnesium | Lubricant | 0.48 | 0.51 | 0.60 | 0.52 |
| stearate | | | | | |
| Size 4, Swedish | Capsule | 1 | — | — | — |
| Orange HPMC | Shell | capsule | | | |
| Capsules | | | | | |
| Size 3, Blue | Capsule | — | — | 1 | — |
| Green HPMC | Shell | | | capsule | |
| Capsules | | | | | |
| Total | | 95.00 | 100 | 115.00 | 100 |

The composition of the Swedish orange hypromellose capsule shells is described in Table 2.

TABLE 2

| Composition of capsule | | |
|---|---|---|
| body and cap | Function | Content |
| FDA/E172 RED | Colorant | 1.1817% |
| IRON OXIDE | | |
| TITANIUM DIOXIDE | Opacifier | 0.4916% |
| Hypromellose | Structure | qs 100% |

The composition of blue green hypromellose capsule shells is described in Table 3.

TABLE 3

| Composition of capsule | | |
|---|---|---|
| body and cap | Function | Content |
| FD&C Blue #1 | Colorant | 0.0281% |
| FD&C Yellow #5 | Colorant | 0.0069% |
| TITANIUM DIOXIDE | Opacifier | 2.2306% |
| Hypromellose | Structure | qs 100% |

### EXAMPLES

### Example 1. A Phase 1 Dose Escalation Study of Compound a for the Treatment of Metastatic Uveal Melanoma

A Phase 1 dose escalation clinical trial of Compound A is conducted. The trial is primarily intended to determine the maximum tolerated dose (MTD) and/or recommended phase 2 dose of the compound when administered orally to subjects with metastatic uveal melanoma (UM).

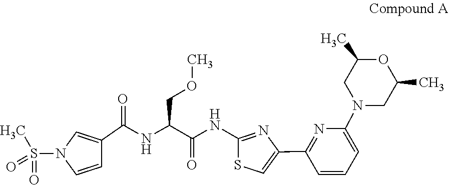 [SMILES: COC[C@H](NC(=O)c1ccn(S(C)(=O)=O)c1)C(=O)Nc1nc(-c2cccc(N3C[C@@H](C)O[C@@H](C)C3)n2)cs1]

In this ongoing study, Compound A has been administered at dose levels of 2.5 mg once a day (QD), 5 mg QD, 7.5 mg QD, and 10 mg QD to 17 subjects with metastatic UM. Preliminary pharmacokinetics (PK) analysis indicates that the compound appears to have a long half-life and that plasma concentrations of the compound increase with increasing dose. One subject treated at the 7.5 mg QD dose level experienced a dose limiting toxicity of Grade 3 keratitis, which improved with a dose hold. In evaluable subjects, myeloid differentiation and reduction in bone marrow and/or peripheral blasts have been observed.

### Example 2. PK Modeling and Simulations of FHD-286 Patient Data Suggest an Optimal Dosing Regimen

A population PK model based on orally administered Compound A dosing data in a total of 10 patients with metastatic uveal melanoma (UM) and advanced hematologic malignancies (e.g., acute myeloid leukemia (AML)) was performed.

Nonlinear mixed-effects models were fitted to concentration vs. time data of FHD-286. Various compartmental models with linear elimination (1-, 2- and 3-compartment), absorption models, structures of omega on clearances, volumes and absorption parameters (diagonal and block) were developed to assess the PK of Compound A.

Overall, a two-compartment model with linear elimination, a zero-order oral absorption, and a lag time of absorption adequately characterized the concentration-time profiles of Compound A.

Exploratory analysis of covariate effects was performed with appropriate plots presenting relationships between the ETAs (e.g., values reflecting the difference between and individual's parameter value and that of the population) of PK parameters to identify any remaining covariate trends. A reduction in clearance (CL) was observed over time (after multiple doses). Therefore a “Multiple Dose Effect” on CL was included in the model to account for this change over time.

Time-varying food effect on the relative bioavailability was also tested a priori in the model but was not significant. No other covariates were included as part of the model. Final PK model parameters are presented in Table 4.

TABLE 4

| | | | BSV % | |
|---|---|---|---|---|
| Parameter | Estimate | RSE % | (Shrinkage %) | |
| Dur (h) | 1.77 | 41.6 | 18.0 | (21.2) |
| Tlag (h) | 0.452 | 0.01 | 19.6 | (21.3) |
| CL (L/h) | 4.07 | 16.2 | 45.6 | (8.6) |
| ×exp(Multiple Dose effect) | −1.35 | 4.8 | | |
| Vc (L) | 54.6 | 43.1 | 77.2 | (16.7) |
| CLp (L/h) | 30.6 | 64.1 | 384 | (9.6) |
| Vd (L) | 165 | 53.2 | 101 | (8.8) |

Error Model

| | | | |
|---|---|---|---|
| Prop Error (%) | 41.1 | 5.8 | N/A |

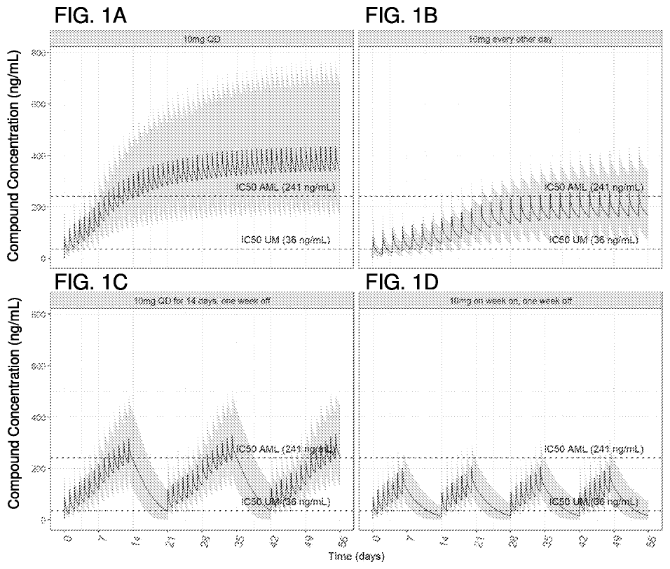

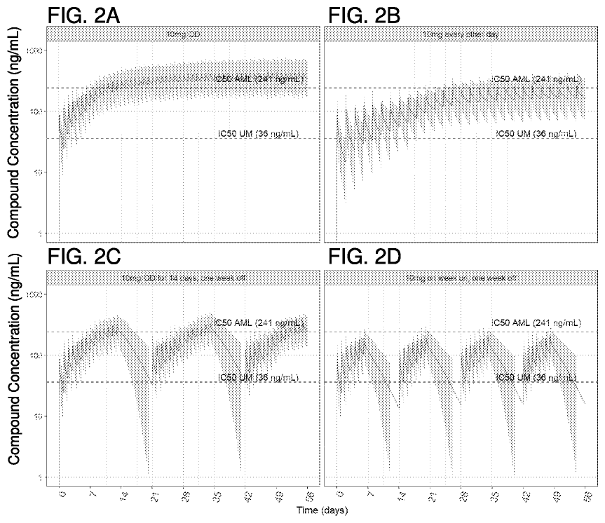

Overall, the model described well Compound A concentrations across the different PK occasions. Based on the PK model, Monte Carlo simulations were performed for 4 different dosing regimen scenarios in a total of 1000 virtual patients (FIGS. 1A-1D and 2A-2D). Compound A concentrations were predicted for 2 dosing cycles of 28 days. Inhibition of CL was considered maximal after 7 doses, and linear regression was used to predict CL inhibition in between. The simulation suggests an optimal dosing regimen of administration of Compound A at least once per day for one to two weeks, followed by one week without administration of Compound A per dosing cycle (FIGS. 1C, 1D, 2C, and 2D).

### OTHER EMBODIMENTS

While the invention has been described in connection with specific embodiments thereof, it will be understood that invention is capable of further modifications and this application is intended to cover any variations, uses, or adaptations of the invention following, in general, the principles of the invention and including such departures from the present disclosure that come within known or customary practice within the art to which the invention pertains and may be applied to the essential features hereinbefore set forth, and follows in the scope of the claims.

Other embodiments are in the claims.

1. A method of treating cancer in a subject in need thereof, the method comprising administering to the subject an effective amount of a compound, N-(1-((4-(6-(2,6-dimethylmorpholino)pyridin-2-yl)thiazol-2-yl)amino)-3-methoxy-1-oxopropan-2-yl)-1-(methylsulfonyl)-1H-pyrrole-3-carboxamide, or a pharmaceutically acceptable salt thereof, at least once daily for six to eight days, followed by six to eight days without administration of the compound, or a pharmaceutically acceptable salt thereof.

2.-6. (canceled)

7. A method of decreasing the level and/or activity of BRG1 and/or BRM in a subject, the method comprising administering to the subject an effective amount of a compound, N-(1-((4-(6-(2,6-dimethylmorpholino)pyridin-2-yl)thiazol-2-yl)amino)-3-methoxy-1-oxopropan-2-yl)-1-(methylsulfonyl)-1H-pyrrole-3-carboxamide, or a pharmaceutically acceptable salt thereof, at least once daily for six to eight days, followed by six to eight days without administration of the compound, or a pharmaceutically acceptable salt thereof.

8.-12. (canceled)

13. The method of claim 7, wherein the subject has cancer.

14. A method of treating a BAF complex-related disorder in a subject in need thereof, the method comprising administering to the subject an effective amount of a compound, N-(1-((4-(6-(2,6-dimethylmorpholino)pyridin-2-yl)thiazol-2-yl)amino)-3-methoxy-1-oxopropan-2-yl)-1-(methylsulfonyl)-1H-pyrrole-3-carboxamide, or a pharmaceutically acceptable salt thereof, at least once daily for six to eight days, followed by six to eight days without administration of the compound, or a pharmaceutically acceptable salt thereof.

15.-25. (canceled)

26. The method of claim 14, wherein the disorder is cancer.

27.-51. (canceled)

52. The method of claim 1, wherein the cancer is metastatic.

53. The method of claim 1, wherein the effective amount is an amount sufficient to reduce cancer tumor growth in the subject compared to a subject that is not administered the compound or a pharmaceutically acceptable salt thereof.

54. The method of claim 1, wherein the effective amount is an amount sufficient to suppress metastatic progression of cancer in the subject compared to a subject that is not administered the compound or a pharmaceutically acceptable salt thereof.

55. The method of claim 1, wherein the effective amount is an amount sufficient to suppress metastatic colonization of cancer in the subject compared to a subject that is not administered the compound or a pharmaceutically acceptable salt thereof.

56. The method of claim 1, wherein the cancer is non-small cell lung cancer, colorectal cancer, bladder cancer, cancer of unknown primary, glioma, breast cancer, melanoma, non-melanoma skin cancer, endometrial cancer, esophagogastric cancer, pancreatic cancer, hepatobiliary cancer, soft tissue sarcoma, ovarian cancer, head and neck cancer, renal cell carcinoma, bone cancer, non-Hodgkin lymphoma, small-cell lung cancer, prostate cancer, embryonal tumor, germ cell tumor, cervical cancer, thyroid cancer, salivary gland cancer, gastrointestinal neuroendocrine tumor, uterine sarcoma, gastrointestinal stromal tumor, CNS cancer, thymic tumor, adrenocortical carcinoma, appendiceal cancer, small bowel cancer, penile cancer, bone cancer, or hematologic cancer.

57.-58. (canceled)

59. The method of claim 56, wherein the cancer is melanoma.

60. The method of claim 59, wherein the melanoma is uveal melanoma, mucosal melanoma, or cutaneous melanoma.

61.-62. (canceled)

63. The method of claim 56, wherein the cancer is hematologic cancer.

64. (canceled)

65. The method of claim 63, wherein the hematologic cancer is acute myeloid leukemia or myelodysplastic syndrome.

66.-74. (canceled)

75. The method of claim 1, wherein the compound or a pharmaceutically acceptable salt thereof is administered in a total dose of between about 5.0 mg and about 20 mg per day.

76.-91. (canceled)

92. The method of claim 75, wherein the total dose is administered to the subject once per day.

93. The method of claim 1, wherein the method comprises at least 21 days of treatment.

94.-96. (canceled)

97. The method of claim 1, wherein the compound is: 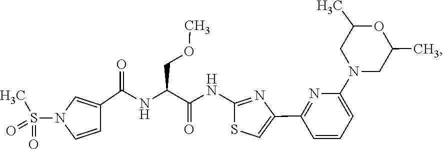 [SMILES: COC[C@H](NC(=O)c1ccn(S(C)(=O)=O)c1)C(=O)Nc1nc(-c2cccc(N3CC(C)OC(C)C3)n2)cs1]
or a pharmaceutically acceptable salt thereof.

98. The method of claim 1, wherein the compound is: 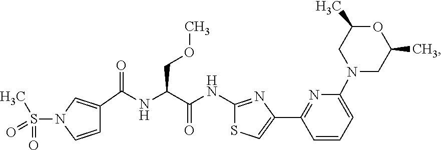 [SMILES: COC[C@H](NC(=O)c1ccn(S(C)(=O)=O)c1)C(=O)Nc1nc(-c2cccc(N3C[C@@H](C)O[C@@H](C)C3)n2)cs1]
or a pharmaceutically acceptable salt thereof.

99. The method of claim 1, wherein the compound is 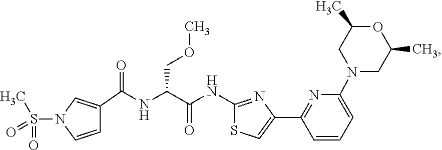 [SMILES: COC[C@@H](NC(=O)c1ccn(S(C)(=O)=O)c1)C(=O)Nc1nc(-c2cccc(N3C[C@@H](C)O[C@@H](C)C3)n2)cs1]
or a pharmaceutically acceptable salt thereof.


SEQUENCE LISTING (4 sequences)

SEQ ID NO: 1 - RNA, 5589 nt, Homo sapiens
  source 1..5589: mol_type = mRNA
ggcgggggaggcgccgggaagtcgacggcgccggcggctcctgcaggaggccactgtctgcagctcccgtgaagatgtccactccagacccacccctgggcggaactcctcggccaggtccttccccgggccctggcccttcccctggagccatgctgggccctagcccgggtccctcgccgggctccgcccacagcatgatggggcccagcccagggccgccctcagcaggacaccccatccccacccaggggcctggagggtaccctcaggacaacatgcaccagatgcacaagcccatggagtccatgcatgagaagggcatgtcggacgacccgcgctacaaccagatgaaaggaatggggatgcggtcagggggccatgctgggatggggcccccgcccagccccatggaccagcactcccaaggttacccctcgcccctgggtggctctgagcatgcctctagtccagttccagccagtggcccgtcttcggggccccagatgtcttccgggccaggaggtgccccgctggatggtgctgacccccaggccttggggcagcagaaccggggcccaaccccatttaaccagaaccagctgcaccagctcagagctcagatcatggcctacaagatgctggccagggggcagcccctccccgaccacctgcagatggcggtgcagggcaagcggccgatgcccgggatgcagcagcagatgccaacgctacctccaccctcggtgtccgcaacaggacccggccctggccctggccctggccccggcccgggtcccggcccggcacctccaaattacagcaggcctcatggtatgggagggcccaacatgcctcccccaggaccctcgggcgtgccccccgggatgccaggccagcctcctggagggcctcccaagccctggcctgaaggacccatggcgaatgctgctgcccccacgagcacccctcagaagctgattcccccgcagccaacgggccgcccttcccccgcgccccctgccgtcccacccgccgcctcgcccgtgatgccaccgcagacccagtcccccgggcagccggcccagcccgcgcccatggtgccactgcaccagaagcagagccgcatcacccccatccagaagccgcggggcctcgaccctgtggagatcctgcaggagcgcgagtacaggctgcaggctcgcatcgcacaccgaattcaggaacttgaaaaccttcccgggtccctggccggggatttgcgaaccaaagcgaccattgagctcaaggccctcaggctgctgaacttccagaggcagctgcgccaggaggtggtggtgtgcatgcggagggacacagcgctggagacagccctcaatgctaaggcctacaagcgcagcaagcgccagtccctgcgcgaggcccgcatcactgagaagctggagaagcagcagaagatcgagcaggagcgcaagcgccggcagaagcaccaggaatacctcaatagcattctccagcatgccaaggatttcaaggaatatcacagatccgtcacaggcaaaatccagaagctgaccaaggcagtggccacgtaccatgccaacacggagcgggagcagaagaaagagaacgagcggatcgagaaggagcgcatgcggaggctcatggctgaagatgaggaggggtaccgcaagctcatcgaccagaagaaggacaagcgcctggcctacctcttgcagcagacagacgagtacgtggctaacctcacggagctggtgcggcagcacaaggctgcccaggtcgccaaggagaaaaagaagaaaaagaaaaagaagaaggcagaaaatgcagaaggacagacgcctgccattgggccggatggcgagcctctggacgagaccagccagatgagcgacctcccggtgaaggtgatccacgtggagagtgggaagatcctcacaggcacagatgcccccaaagccgggcagctggaggcctggctcgagatgaacccggggtatgaagtagctccgaggtctgatagtgaagaaagtggctcagaagaagaggaagaggaggaggaggaagagcagccgcaggcagcacagcctcccaccctgcccgtggaggagaagaagaagattccagatccagacagcgatgacgtctctgaggtggacgcgcggcacatcattgagaatgccaagcaagatgtcgatgatgaatatggcgtgtcccaggcccttgcacgtggcctgcagtcctactatgccgtggcccatgctgtcactgagagagtggacaagcagtcagcgcttatggtcaatggtgtcctcaaacagtaccagatcaaaggtttggagtggctggtgtccctgtacaacaacaacctgaacggcatcctggccgacgagatgggcctggggaagaccatccagaccatcgcgctcatcacgtacctcatggagcacaaacgcatcaatgggcccttcctcatcatcgtgcctctctcaacgctgtccaactgggcgtacgagtttgacaagtgggccccctccgtggtgaaggtgtcttacaagggatccccagcagcaagacgggcctttgtcccccagctccggagtgggaagttcaacgtcttgctgacgacgtacgagtacatcatcaaagacaagcacatcctcgccaagatccgttggaagtacatgattgtggacgaaggtcaccgcatgaagaaccaccactgcaagctgacgcaggtgctcaacacgcactatgtggcaccccgccgcctgctgctgacgggcacaccgctgcagaacaagcttcccgagctctgggcgctgctcaacttcctgctgcccaccatcttcaagagctgcagcaccttcgagcagtggtttaacgcaccctttgccatgaccggggaaaaggtggacctgaatgaggaggaaaccattctcatcatccggcgtctccacaaagtgctgcggcccttcttgctccgacgactcaagaaggaagtcgaggcccagttgcccgaaaaggtggagtacgtcatcaagtgcgacatgtctgcgctgcagcgagtgctctaccgccacatgcaggccaagggcgtgctgctgactgatggctccgagaaggacaagaagggcaaaggcggcaccaagaccctgatgaacaccatcatgcagctgcggaagatctgcaaccacccctacatgttccagcacatcgaggagtccttttccgagcacttggggttcactggcggcattgtccaagggctggacctgtaccgagcctcgggtaaatttgagcttcttgatagaattcttcccaaactccgagcaaccaaccacaaagtgctgctgttctgccaaatgacctccctcatgaccatcatggaagattactttgcgtatcgcggctttaaatacctcaggcttgatggaaccacgaaggcggaggaccggggcatgctgctgaaaaccttcaacgagcccggctctgagtacttcatcttcctgctcagcacccgggctggggggctcggcctgaacctccagtcggcagacactgtgatcatttttgacagcgactggaatcctcaccaggacctgcaagcgcaggaccgagcccaccgcatcgggcagcagaacgaggtgcgtgtgctccgcctctgcaccgtcaacagcgtggaggagaagatcctagctgcagccaagtacaagctcaacgtggaccagaaggtgatccaggccggcatgttcgaccagaagtcctccagccatgagcggcgcgccttcctgcaggccatcctggagcacgaggagcaggatgagagcagacactgcagcacgggcagcggcagtgccagcttcgcccacactgcccctccgccagcgggcgtcaaccccgacttggaggagccacctctaaaggaggaagacgaggtgcccgacgacgagaccgtcaaccagatgatcgcccggcacgaggaggagtttgatctgttcatgcgcatggacctggaccgcaggcgcgaggaggcccgcaaccccaagcggaagccgcgcctcatggaggaggacgagctcccctcgtggatcatcaaggacgacgcggaggtggagcggctgacctgtgaggaggaggaggagaagatgttcggccgtggctcccgccaccgcaaggaggtggactacagcgactcactgacggagaagcagtggctcaagaaaattacaggaaaagatatccatgacacagccagcagtgtggcacgtgggctacaattccagcgtggccttcagttctgcacacgtgcgtcaaaggccatcgaggagggcacgctggaggagatcgaagaggaggtccggcagaagaaatcatcacggaagcgcaagcgagacagcgacgccggctcctccaccccgaccaccagcacccgcagccgcgacaaggacgacgagagcaagaagcagaagaagcgcgggcggccgcctgccgagaaactctcccctaacccacccaacctcaccaagaagatgaagaagattgtggatgccgtgatcaagtacaaggacagcagcagtggacgtcagctcagcgaggtcttcatccagctgccctcgcgaaaggagctgcccgagtactacgagctcatccgcaagcccgtggacttcaagaagataaaggagcgcattcgcaaccacaagtaccgcagcctcaacgacctagagaaggacgtcatgctcctgtgccagaacgcacagaccttcaacctggagggctccctgatctatgaagactccatcgtcttgcagtcggtcttcaccagcgtgcggcagaaaatcgagaaggaggatgacagtgaaggcgaggagagtgaggaggaggaagagggcgaggaggaaggctccgaatccgaatctcggtccgtcaaagtgaagatcaagcttggccggaaggagaaggcacaggaccggctgaagggcggccggcggcggccgagccgagggtcccgagccaagccggtcgtgagtgacgatgacagtgaggaggaacaagaggaggaccgctcaggaagtggcagcgaagaagactgagccccgacattccagtctcgaccccgagcccctcgttccagagctgagatggcataggccttagcagtaacgggtagcagcagatgtagtttcagacttggagtaaaactgtataaacaaaagaatcttccatatttatacagcagagaagctgtaggactgtttgtgactggccctgtcctggcatcagtagcatctgtaacagcattaactgtcttaaagagagagagagagaattccgaattggggaacacacgatacctgtttttcttttccgttgctggcagtactgttgcgccgcagtttggagtcactgtagttaagtgtggatgcatgtgcgtcaccgtccactcctcctactgtattttattggacaggtcagactcgccgggggcccggcgagggtatgtcagtgtcactggatgtcaaacagtaataaattaaaccaacaacaaaacgcacagccaaaaaaaaa

SEQ ID NO: 2 - protein, 1647 aa, Homo sapiens
MSTPDPPLGGTPRPGPSPGPGPSPGAMLGPSPGPSPGSAHSMMGPSPGPPSAGHPIPTQGPGGYPQDNMHQMHKPMESMHEKGMSDDPRYNQMKGMGMRSGGHAGMGPPPSPMDQHSQGYPSPLGGSEHASSPVPASGPSSGPQMSSGPGGAPLDGADPQALGQQNRGPTPFNQNQLHQLRAQIMAYKMLARGQPLPDHLQMAVQGKRPMPGMQQQMPTLPPPSVSATGPGPGPGPGPGPGPGPAPPNYSRPHGMGGPNMPPPGPSGVPPGMPGQPPGGPPKPWPEGPMANAAAPTSTPQKLIPPQPTGRPSPAPPAVPPAASPVMPPQTQSPGQPAQPAPMVPLHQKQSRITPIQKPRGLDPVEILQEREYRLQARIAHRIQELENLPGSLAGDLRTKATIELKALRLLNFQRQLRQEVVVCMRRDTALETALNAKAYKRSKRQSLREARITEKLEKQQKIEQERKRRQKHQEYLNSILQHAKDFKEYHRSVTGKIQKLTKAVATYHANTEREQKKENERIEKERMRRLMAEDEEGYRKLIDQKKDKRLAYLLQQTDEYVANLTELVRQHKAAQVAKEKKKKKKKKKAENAEGQTPAIGPDGEPLDETSQMSDLPVKVIHVESGKILTGTDAPKAGQLEAWLEMNPGYEVAPRSDSEESGSEEEEEEEEEEQPQAAQPPTLPVEEKKKIPDPDSDDVSEVDARHIIENAKQDVDDEYGVSQALARGLQSYYAVAHAVTERVDKQSALMVNGVLKQYQIKGLEWLVSLYNNNLNGILADEMGLGKTIQTIALITYLMEHKRINGPFLIIVPLSTLSNWAYEFDKWAPSVVKVSYKGSPAARRAFVPQLRSGKFNVLLTTYEYIIKDKHILAKIRWKYMIVDEGHRMKNHHCKLTQVLNTHYVAPRRLLLTGTPLQNKLPELWALLNFLLPTIFKSCSTFEQWFNAPFAMTGEKVDLNEEETILIIRRLHKVLRPFLLRRLKKEVEAQLPEKVEYVIKCDMSALQRVLYRHMQAKGVLLTDGSEKDKKGKGGTKTLMNTIMQLRKICNHPYMFQHIEESFSEHLGFTGGIVQGLDLYRASGKFELLDRILPKLRATNHKVLLFCQMTSLMTIMEDYFAYRGFKYLRLDGTTKAEDRGMLLKTFNEPGSEYFIFLLSTRAGGLGLNLQSADTVIIFDSDWNPHQDLQAQDRAHRIGQQNEVRVLRLCTVNSVEEKILAAAKYKLNVDQKVIQAGMFDQKSSSHERRAFLQAILEHEEQDESRHCSTGSGSASFAHTAPPPAGVNPDLEEPPLKEEDEVPDDETVNQMIARHEEEFDLFMRMDLDRRREEARNPKRKPRLMEEDELPSWIIKDDAEVERLTCEEEEEKMFGRGSRHRKEVDYSDSLTEKQWLKAIEEGTLEEIEEEVRQKKSSRKRKRDSDAGSSTPTTSTRSRDKDDESKKQKKRGRPPAEKLSPNPPNLTKKMKKIVDAVIKYKDSSSGRQLSEVFIQLPSRKELPEYYELIRKPVDFKKIKERIRNHKYRSLNDLEKDVMLLCQNAQTFNLEGSLIYEDSIVLQSVFTSVRQKIEKEDDSEGEESEEEEEGEEEGSESESRSVKVKIKLGRKEKAQDRLKGGRRRPSRGSRAKPVVSDDDSEEEQEEDRSGSGSEED

SEQ ID NO: 3 - RNA, 5892 nt, Homo sapiens
  source 1..5892: mol_type = mRNA
gcgtcttccggcgcccgcggaggaggcgagggtgggacgctgggcggagcccgagtttaggaagaggaggggacggctgtcatcaatgaagtcatattcataatctagtcctctctccctctgtttctgtactctgggtgactcagagagggaagagattcagccagcacactcctcgcgagcaagcattactctactgactggcagagacaggagaggtagatgtccacgcccacagaccctggtgcgatgccccacccagggccttcgccggggcctgggccttcccctgggccaattcttgggcctagtccaggaccaggaccatccccaggttccgtccacagcatgatggggccaagtcctggacctccaagtgtctcccatcctatgccgacgatggggtccacagacttcccacaggaaggcatgcatcaaatgcataagcccatcgatggtatacatgacaaggggattgtagaagacatccattgtggatccatgaagggcactggtatgcgaccacctcacccaggcatgggccctccccagagtccaatggatcaacacagccaaggttatatgtcaccacacccatctccattaggagccccagagcacgtctccagccctatgtctggaggaggcccaactccacctcagatgccaccaagccagccgggggccctcatcccaggtgatccgcaggccatgagccagcccaacagaggtccctcacctttcagtcctgtccagctgcatcagcttcgagctcagattttagcttataaaatgctggcccgaggccagcccctccccgaaacgctgcagcttgcagtccaggggaaaaggacgttgcctggcttgcagcaacaacagcagcagcaacagcagcagcagcagcagcagcagcagcagcagcagcagcaacagcagccgcagcagcagccgccgcaaccacagacgcagcaacaacagcagccggcccttgttaactacaacagaccatctggcccggggccggagctgagcggcccgagcaccccgcagaagctgccggtgcccgcgcccggcggccggccctcgcccgcgccccccgcagccgcgcagccgcccgcggccgcagtgcccgggccctcagtgccgcagccggccccggggcagccctcgcccgtcctccagctgcagcagaagcagagccgcatcagccccatccagaaaccgcaaggcctggaccccgtggaaattctgcaagagcgggaatacagacttcaggcccgcatagctcataggatacaagaactggaaaatctgcctggctctttgccaccagatttaagaaccaaagcaaccgtggaactaaaagcacttcggttactcaatttccagcgtcagctgagacaggaggtggtggcctgcatgcgcagggacacgaccctggagacggctctcaactccaaagcatacaaacggagcaagcgccagactctgagagaagctcgcatgaccgagaagctggagaagcagcagaagattgagcaggagaggaaacgccgtcagaaacaccaggaatacctgaacagtattttgcaacatgcaaaagattttaaggaatatcatcggtctgtggccggaaagatccagaagctctccaaagcagtggcaacttggcatgccaacactgaaagagagcagaagaaggagacagagcggattgaaaaggagagaatgcggcgactgatggctgaagatgaggagggttatagaaaactgattgatcaaaagaaagacaggcgtttagcttaccttttgcagcagaccgatgagtatgtagccaatctgaccaatctggtttgggagcacaagcaagcccaggcagccaaagagaagaagaagaggaggaggaggaagaagaaggctgaggagaatgcagagggtggggagtctgccctgggaccggatggagagcccatagatgagagcagccagatgagtgacctccctgtcaaagtgactcacacagaaaccggcaaggttctgttcggaccagaagcacccaaagcaagtcagctggacgcctggctggaaatgaatcctggttatgaagttgcccctagatctgacagtgaagagagtgattctgattatgaggaagaggatgaggaagaagagtccagtaggcaggaaaccgaagagaaaatactcctggatccaaatagcgaagaagtttctgagaaggatgctaagcagatcattgagacagctaagcaagacgtggatgatgaatacagcatgcagtacagtgccaggggctcccagtcctactacaccgtggctcatgccatctcggagagggtggagaaacagtctgccctcctaattaatgggaccctaaagcattaccagctccagggcctggaatggatggtttccctgtataataacaacttgaacggaatcttagccgatgaaatggggcttggaaagaccatacagaccattgcactcatcacttatctgatggagcacaaaagactcaatggcccctatctcatcattgttcccctttcgactctatctaactggacatatgaatttgacaaatgggctccttctgtggtgaagatttcttacaagggtactcctgccatgcgtcgctcccttgtcccccagctacggagtggcaaattcaatgtcctcttgactacttatgagtatattataaaagacaagcacattcttgcaaagattcggtggaaatacatgatagtggacgaaggccaccgaatgaagaatcaccactgcaagctgactcaggtcttgaacactcactatgtggcccccagaaggatcctcttgactgggaccccgctgcagaataagctccctgaactctgggccctcctcaacttcctcctcccaacaatttttaagagctgcagcacatttgaacaatggttcaatgctccatttgccatgactggtgaaagggtggacttaaatgaagaagaaactatattgatcatcaggcgtctacataaggtgttaagaccatttttactaaggagactgaagaaagaagttgaatcccagcttcccgaaaaagtggaatatgtgatcaagtgtgacatgtcagctctgcagaagattctgtatcgccatatgcaagccaaggggatccttctcacagatggttctgagaaagataagaaggggaaaggaggtgctaagacacttatgaacactattatgcagttgagaaaaatctgcaaccacccatatatgtttcagcacattgaggaatcctttgctgaacacctaggctattcaaatggggtcatcaatggggctgaactgtatcgggcctcagggaagtttgagctgcttgatcgtattctgccaaaattgagagcgactaatcaccgagtgctgcttttctgccagatgacatctctcatgaccatcatggaggattattttgcttttcggaacttcctttacctacgccttgatggcaccaccaagtctgaagatcgtgctgctttgctgaagaaattcaatgaacctggatcccagtatttcattttcttgctgagcacaagagctggtggcctgggcttaaatcttcaggcagctgatacagtggtcatctttgacagcgactggaatcctcatcaggatctgcaggcccaagaccgagctcaccgcatcgggcagcagaacgaggtccgggtactgaggctctgtaccgtgaacagcgtggaggaaaagatcctcgcggccgcaaaatacaagctgaacgtggatcagaaagtgatccaggcgggcatgtttgaccaaaagtcttcaagccacgagcggagggcattcctgcaggccatcttggagcatgaggaggaaaatgaggaagaagatgaagtaccggacgatgagactctgaaccaaatgattgctcgacgagaagaagaatttgacctttttatgcggatggacatggaccggcggagggaagatgcccggaacccgaaacggaagccccgtttaatggaggaggatgagctgccctcctggatcattaaggatgacgctgaagtagaaaggctcacctgtgaagaagaggaggagaaaatatttgggagggggtcccgccagcgccgtgacgtggactacagtgacgccctcacggagaagcagtggctaagggccatcgaagacggcaatttggaggaaatggaagaggaagtacggcttaagaagcgaaaaagacgaagaaatgtggataaagatcctgcaaaagaagatgtggaaaaagctaagaagagaagaggccgccctcccgctgagaaactgtcaccaaatccccccaaactgacaaagcagatgaacgctatcatcgatactgtgataaactacaaagataggtgtaacgtggagaaggtgcccagtaattctcagttggaaatagaaggaaacagttcagggcgacagctcagtgaagtcttcattcagttaccttcaaggaaagaattaccagaatactatgaattaattaggaagccagtggatttcaaaaaaataaaggaaaggattcgtaatcataagtaccggagcctaggcgacctggagaaggatgtcatgcttctctgtcacaacgctcagacgttcaacctggagggatcccagatctatgaagactccatcgtcttacagtcagtgtttaagagtgcccggcagaaaattgccaaagaggaagagagtgaggatgaaagcaatgaagaggaggaagaggaagatgaagaagagtcagagtccgaggcaaaatcagtcaaggtgaaaattaagctcaataaaaaagatgacaaaggccgggacaaagggaaaggcaagaaaaggccaaatcgaggaaaagccaaacctgtagtgagcgattttgacagcgatgaggagcaggatgaacgtgaacagtcagaaggaagtgggacggatgatgagtgatcagtatggacctttttccttggtagaactgaattccttcctcccctgtctcatttctacccagtgagttcatttgtcatataggcactgggttgtttctatatcatcatcgtctataaactagctttaggatagtgccagacaaacatatgatatcatggtgtaaaaaacacacacatacacaaatatttgtaacatattgtgaccaaatgggcctcaaagattcagattgaaacaaacaaaaagcttttgatggaaaatatgtgggtggatagtatatttctatgggtgggtctaatttggtaacggtttgattgtgcctggttttatcacctgttcagatgagaagatttttgtcttttgtagcactgataaccaggagaagccattaaaagccactggttattttatttttcatcaggcaattttcgaggtttttatttgttcggtattgtttttttacactgtggtacatataagcaactttaataggtgataaatgtacagtagttagatttcacctgcatatacatttttccattttatgctctatgatctgaacaaaagctttttgaattgtataagatttatgtctactgtaaacattgcttaatttttttgctcttgatttaaaaaaaagttttgttgaaagcgctattgaatattgcaatctatatagtgtattggatggcttcttttgtcaccctgatctcctatgttaccaatgtgtatcgtctccttctccctaaagtgtacttaatctttgctttctttgcacaatgtctttggttgcaagtcataagcctgaggcaaataaaattccagtaatttcgaagaatgtggtgttggtgctttcctaataaagaaataatttagcttgacaaaaaaaaaaaaaaa

SEQ ID NO: 4 - protein, 1590 aa, Homo sapiens
MSTPTDPGAMPHPGPSPGPGPSPGPILGPSPGPGPSPGSVHSMMGPSPGPPSVSHPMPTMGSTDFPQEGMHQMHKPIDGIHDKGIVEDIHCGSMKGTGMRPPHPGMGPPQSPMDQHSQGYMSPHPSPLGAPEHVSSPMSGGGPTPPQMPPSQPGALIPGDPQAMSQPNRGPSPFSPVQLHQLRAQILAYKMLARGQPLPETLQLAVQGKRTLPGLQQQQQQQQQQQQQQQQQQQQQQQPQQQPPQPQTQQQQQPALVNYNRPSGPGPELSGPSTPQKLPVPAPGGRPSPAPPAAAQPPAAAVPGPSVPQPAPGQPSPVLQLQQKQSRISPIQKPQGLDPVEILQEREYRLQARIAHRIQELENLPGSLPPDLRTKATVELKALRLLNFQRQLRQEVVACMRRDTTLETALNSKAYKRSKRQTLREARMTEKLEKQQKIEQERKRRQKHQEYLNSILQHAKDFKEYHRSVAGKIQKLSKAVATWHANTEREQKKETERIEKERMRRLMAEDEEGYRKLIDQKKDRRLAYLLQQTDEYVANLTNLVWEHKQAQAAKEKKKRRRRKKKAEENAEGGESALGPDGEPIDESSQMSDLPVKVTHTETGKVLFGPEAPKASQLDAWLEMNPGYEVAPRSDSEESDSDYEEEDEEEESSRQETEEKILLDPNSEEVSEKDAKQIIETAKQDVDDEYSMQYSARGSQSYYTVAHAISERVEKQSALLINGTLKHYQLQGLEWMVSLYNNNLNGILADEMGLGKTIQTIALITYLMEHKRLNGPYLIIVPLSTLSNWTYEFDKWAPSVVKISYKGTPAMRRSLVPQLRSGKFNVLLTTYEYIIKDKHILAKIRWKYMIVDEGHRMKNHHCKLTQVLNTHYVAPRRILLTGTPLQNKLPELWALLNFLLPTIFKSCSTFEQWFNAPFAMTGERVDLNEEETILIIRRLHKVLRPFLLRRLKKEVESQLPEKVEYVIKCDMSALQKILYRHMQAKGILLTDGSEKDKKGKGGAKTLMNTIMQLRKICNHPYMFQHIEESFAEHLGYSNGVINGAELYRASGKFELLDRILPKLRATNHRVLLFCQMTSLMTIMEDYFAFRNFLYLRLDGTTKSEDRAALLKKFNEPGSQYFIFLLSTRAGGLGLNLQAADTVVIFDSDWNPHQDLQAQDRAHRIGQQNEVRVLRLCTVNSVEEKILAAAKYKLNVDQKVIQAGMFDQKSSSHERRAFLQAILEHEEENEEEDEVPDDETLNQMIARREEEFDLFMRMDMDRRREDARNPKRKPRLMEEDELPSWIIKDDAEVERLTCEEEEEKIFGRGSRQRRDVDYSDALTEKQWLRAIEDGNLEEMEEEVRLKKRKRRRNVDKDPAKEDVEKAKKRRGRPPAEKLSPNPPKLTKQMNAIIDTVINYKDRCNVEKVPSNSQLEIEGNSSGRQLSEVFIQLPSRKELPEYYELIRKPVDFKKIKERIRNHKYRSLGDLEKDVMLLCHNAQTFNLEGSQIYEDSIVLQSVFKSARQKIAKEEESEDESNEEEEEEDEEESESEAKSVKVKIKLNKKDDKGRDKGKGKKRPNRGKAKPVVSDFDSDEEQDEREQSEGSGTDDE